(19) United States
(12) Patent Application Publication
(10) Pub. No.: US 2016/0103138 A1
(43) Pub. Date: Apr. 14, 2016

(54) METHODS FOR ANALYZING ELASTIC FIBER INJURY MARKERS AND DIAGNOSIS OF DISEASES ASSOCIATED WITH ELASTIC FIBER INJURY

(71) Applicants: Yong Y. LIN, Bridgewater, NJ (US); Shuren MA, Cliffside Park, NJ (US); Gerard M. TURINO, New York, NY (US); Toyonobu USUKI, Tokyo (JP)

(72) Inventors: Yong Y. Lin, Bridgewater, NJ (US); Shuren Ma, Cliffside Park, NJ (US); Gerard M. Turino, New York, NY (US); Toyonobu Usuki, Setagaya Tokyo (JP)

(21) Appl. No.: 14/888,075

(22) PCT Filed: Apr. 30, 2014
(86) PCT No.: PCT/US14/36070

§ 371 Date: Oct. 29, 2015

Related U.S. Application Data
(60) Provisional application No. 61/817,669, filed on Apr. 30, 2013.

(51) Int. Cl. G01N 33/68; A61K 38/57; G01N 30/72

(52) U.S. Cl. CPC G01N 33/6887; G01N 30/7233; A61K 38/57; G01N 2333/78; G01N 2800/122

(57) ABSTRACT

The present invention provides, inter alia, methods for measuring the amount of a marker of elastic fiber injury in a sample. The methods include contacting a sample with a compound of formula (1) and carrying out mass spectrometry on the sample containing the compound of formula (1). Also provided are methods of diagnosing whether a subject has a disease characterized by elastic fiber injury, a method for improving the accuracy and precision of mass spectroscopy analysis of a marker of elastic fiber injury, and kits for determining, by mass spectrometry, the amount of a marker of elastic fiber injury in a sample from a subject. Further provided are methods for preventing the progression of the effects associated with alpha-1 antitrypsin deficiency (AATD) in a subject with normal lung function.

### CROSS-REFERENCE TO RELATED APPLICATIONS

The present invention claims benefit to U.S. Provisional Application No. 61/817,669 filed Apr. 30, 2013. The entire contents of the above application are incorporated by reference.

### FIELD OF INVENTION

The present invention provides, inter alia, methods for measuring the amount of a marker of elastic fiber injury in a sample. Also provided are methods of diagnosing whether a subject has a disease characterized by elastic fiber injury, methods for preventing the progression of the effects associated with alpha-1 antitrypsin deficiency (AATD) in a subject with normal lung function, methods for improving the accuracy and precision of mass spectroscopy analysis of a marker of elastic fiber injury, and kits for determining, by mass spectrometry, the amount of a marker of elastic fiber injury in a sample from a subject.

### BACKGROUND OF THE INVENTION

Elastic fibers are significant structural constituents of skin, blood vessels, and lungs, where they provide physical recoil to distorting forces and contribute to normal physiological function. (Mecham, R. P. et al., 1997). Elastin, a major structural component of elastic fibers, is a highly crosslinked insoluble protein formed by post-translational modification of lysine resides in the soluble precursor, tropoelastin (786 amino acids), by lysyl oxidase and condensation reactions. Desmosine (DES) and isodesmosine (IDS) are two unique pyridinium amino acids that serve as major crosslinking molecules binding the polymeric chains of amino acids into the 3D network of elastin. (Thomas, J. et al., 1963, Shimada, W. et al., 1969, Akagawa, M. et al., 2000).

The degradation of elastin-containing tissues that occurs in several widely prevalent diseases (Sandberg, L. B. et al., 1981, Rosenbloom, J. et al., 1982), such as atherosclerosis (Galis, Z. S. et al., 1994, Dollery, C. M. et al., 2003, Umeda, H. et al., 2011), aortic aneurysms (Watanabe, M. et al., 1999, Marque, V. et al., 2001), skin lesions (Schwartz, E. et al., 1990, Annovazzi, L., Viglio, S., Gheduzzi, D. et al., 2004), cystic fibrosis (Viglio, S. et al., 2000, Stone, P. J., Konstan, M. W. et al., 1995), and chronic obstructive pulmonary disease (COPD) which includes pulmonary emphysema, etc. (Schriver, E. E. et al., 1992, Tenholder, M. F. et al., 1991, Stone, P. J., Gottlieb, D. J. et al., 1995, Bode, D. C. et al., 2000, Boschetto, P. et al., 2006, Luisetti, M. et al., 2008, Ma, S. et al., 2003, Ma, S. et al., 2007), have been associated with increased excretion in the body fluids of peptides containing these two pyridinium compounds.

For example, alpha-1 antitrypsin deficiency (AATD) is a genetic cause of COPD that affects as many as 100,000 Americans. (Brantly et al., 1988) Studies of the natural history of severe AATD suggest that forced expiratory volume in one second (FEV₁) is an imperfect marker of disease presence and progression. (Dirksen et al., 1997). Recent studies have shown that quantitative chest computed tomography (QCT) scans are more sensitive than pulmonary function tests at detecting the lung parenchymal destruction that occurs in many AATD individuals. (Ma et al., 2013). Accordingly, better methods of detecting lung elastin degradation are needed.

Furthermore, augmentation of circulatory levels of alpha-1 antitrypsin (AAT) protein has been a prescribed therapy for the long-term treatment of severe AATD for over 25 years. (Wewers et al., 1987). The hypothesis is that maintaining higher levels of alpha-1 protein in blood and tissues should be protective against the effect of neutrophil elastase, for which AAT is the major systemic inhibitor. However, attempts to demonstrate a positive effect on elastin degradation by augmentation therapy have been inconsistent.

Stone and colleagues studied two AATD patients, a 63 year old female with an FEV₁ of 72% predicted and a 41 year old male with FEV₁ of 45% predicted. They received monthly infusions of 260 mg/kg of AAT and were followed for 18 months. Mean values of post-treatment urinary desmosine values determined by the isotope-dilution high performance liquid chromatography (HPLC) method show a sustained drop that exceeded 35% in both subjects from pre-treatment levels. (Stone et al., 1995). Measurements of desmosine in body fluids other than urine were not available in this study.

A study was carried out by the American-Italian study group in 2000. (Gottlieb et al., 2000) This trial was unblinded and open label, and studied 12 AATD subjects (8 men and 4 women; genotypes 11 PIZZ and one PI Mprocida/Mprocida) with severe to moderate emphysema (baseline FEV₁ 41±19% predicted) who received supplementation with Prolastin (Bayer Company) with a weekly regimen of 60 mg/kg for 4 weeks. Spot urine samples were collected weekly for 4 weeks during the run in and then weekly prior to each of the weekly infusions (plasma 12 specimens). Further urine specimens were collected 2 days after the infusion in weeks 2 and 4 (peak specimens). Urinary desmosine values were determined by the isotope-dilution HPLC method. (Stone et al., 1991).

During supplementation, the urinary desmosine excretion was unchanged in comparison with the run in. In this study the AATD subjects with emphysema never receiving supplementation therapy excreted more desmosine than healthy smokers or COPD patients with normal AAT, a result consistent with higher plasma levels of DI in AATD patients than in non-AATD COPD patients demonstrated recently. (Ma et al., 2007).

In 2002, Stoller et al. reported a randomized controlled trial with 26 AATD subjects to evaluate the bioequivalence of 2 commercially available preparations of pooled human plasma AAT. Patients were studied for 24 weeks and urinary desmosine excretion was measured weekly by 2 methods, the isotope-dilution HPLC method (Stone et al., 1991), and RIA method. (King et al., 1980). Desmosine values showed a good correlation between the 2 methods of measurement but no significant differences occurred between values at entry and after 24 weeks of treatment.

Changes were introduced in the methods of analysis of desmosine and isodesmosine (DI) using mass spectrometry in 2003. (Ma et al., 2003). This increased specificity and sensitivity for such measurements in body fluids, which included plasma and sputum as well as urine. This method has recently been modified to include an acetylated pyridinoline internal standard, which improves accuracy and was applied in this study. (Ma et al., 2011).

A free, non-conjugated protein component of DI in urine is measurable by mass spectrometry and is an indicator of increased elastin peptide degradation in vivo prior to excretion and consistent with an increase in elastase activity. (Rodriguez et al., 1979).

In 2007, results of measurements of DI in urine, plasma and sputum in patients with COPD related to AATD and COPD patients with normal levels of AAT were published. (Ma et al., 2007). In both groups the levels of DI in plasma and sputum and the unconjugated free component of DI in urine were significantly elevated above control values. The patients with AATD had values of DI which were significantly above the non-AATD patients. All the AATD patients were on augmentation therapy. Pre-augmentation therapy values were not available in this population of patients. The availability of fluid samples for analysis of plasma, bronchoalveolar lavage fluid (BALF) and urine before starting augmentation and again after augmentation therapy allowed such an analysis.

Several methodologies for measuring DES and IDS have been developed in the last two decades. These include enzymatic immunoassay (ELISA) (Cocci, F. et al., 2002, Luisetti, M. et al., 1996), radioimmunoassay (McClintock, D. E. et al., 2006, Starcher, B. et al., 1995), capillary electrophoresis (Viglio, S. et al., 2000, Annovazzi, L., Viglio, S., Perani, E. et al., 2004, Fiorenza, D. et al., 2002, Stolk, J. et al., 2005), high-performance liquid chromatography (HPLC) (Stone, P. J., Gottlieb, D. J. et al., 1995, Stone, P. J., Konstan, M. W. et al., 1995 (2), Stone, P. J. et al., 1991, Cumiskey, W. R. et al., 1995), electrokinetic chromatography (Viglio, S. et al., 1998), and liquid chromatography mass spectrometry (LC-MS) or tandem mass spectrometry (LC-MS/MS) (Ma, S. et al., 2003, Ma, S. et al., 2007, Boutin, M. et al., 2009 (1), Albarbarawi, O. et al., 2010, Ma, S. et al. 2011).

Among these methodologies the LC-MS/MS method is believed to provide the most sensitivity and specificity. Since DES and IDS are present in body fluids in extremely low concentrations, their precise and specific measurements have been a challenge. Two major improvements in the LC-MS/MS method have been made recently. One is by Albarbarawi, O. et al. that introduced a catalytically exchanged deuterium DES as the internal standard (IS) for urinary total DES+IDS analysis (Albarbarawi, O. et al., 2010). The second is by Ma, S. et al., (2011), which used acetylated pyridinoline as the IS to DES and IDS in several types of body fluids (urine, plasma, sputum, etc.). (Ma, S. et al., 2011).

In sum, better methods for measuring the amount of a marker of elastic fiber injury and for detecting lung elastin degradation are needed. The present application is directed to, inter alia, meeting these needs.

### SUMMARY OF THE INVENTION

We have now found that the ISs used in the above methods are not stable enough toward acid hydrolysis, which is the analytical step required to release the crosslinking DES and IDS molecules in a sample.

Recently we have succeeded in the total chemical synthesis of the DES molecule (Usuki, T. et al., 2012, Yanuma, H. et al., 2012). Subsequently, we have synthesized a stable deuterate molecule, DES-d₄ (FIG. 1), which can serve as an ideal IS for the LC-MS/MS analysis of crosslinking DES and IDS. We report herein the chemical synthesis of DES-d₄ and its application in the isotope dilution LC-MS/MS analysis of DES and IDS, which can be used with a wide variety of biological samples relevant to elastin degradation.

In view of the foregoing, one embodiment of the present invention is a method for measuring the amount of a marker of elastic fiber injury selected from the group consisting of desmosine, isodesmosine, and a combination thereof in a sample. This method comprises contacting the sample with a compound of formula (1):

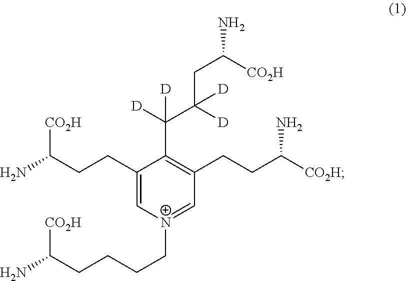 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

and carrying out mass spectrometry on the sample containing the compound of formula (1).

Another embodiment of the present invention is a method for diagnosing whether a subject has a disease characterized by an elastic fiber injury. This method comprises

(a) contacting a compound of formula (1):

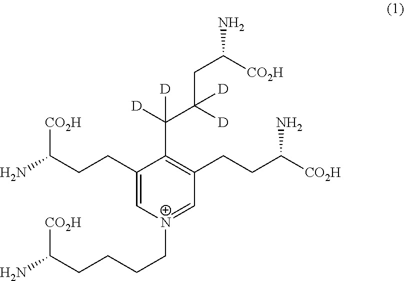 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

with a sample obtained from the subject; and

(b) measuring, by mass spectrometry, the amount of a marker of elastic fiber injury selected from the group consisting of desmosine, isodesmosine, and a combination thereof in the sample.

An additional embodiment of the present invention is a method for improving the accuracy and precision of mass spectroscopy analysis of a marker of elastic fiber injury in a sample, the marker being selected from the group consisting of desmosine, isodesmosine, and a combination thereof. This method comprises:

(a) contacting a compound of formula (1):

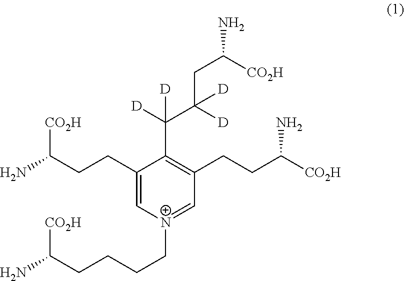 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

with a sample from a subject suspected of having a disease characterized by elastic fiber injury;

(b) carrying out acid hydrolysis of the sample from step (a) containing the compound of formula (1); and

(c) carrying out mass spectrometry on the acid hydrolyzed sample from step (b).

Another embodiment of the present invention is a kit for determining, by mass spectrometry, the amount of a marker of elastic fiber injury in a sample from a subject. The kit comprises a compound of formula (1):

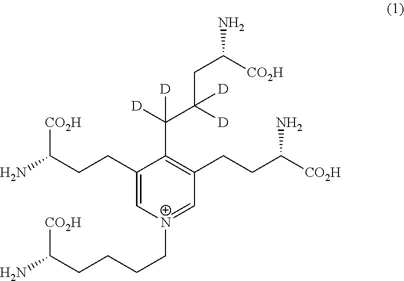 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

and instructions for use thereof, wherein the marker of elastic fiber injury is selected from the group consisting of desmosine, isodesmosine, and a combination thereof.

A further embodiment of the present invention is a method for preventing the progression of the effects associated with alpha-1 antitrypsin deficiency (AATD) in a subject with normal lung function. This method comprises

(a) measuring, by mass spectrometry, a marker of elastic fiber injury selected from the group consisting of desmosine, isodesmosine, and a combination thereof in a sample from the subject; and

(b) administering AATD augmentation therapy if the subject has a higher than normal amount of the marker of elastic fiber injury.

An additional embodiment of the present invention is a method for detecting lung elastin degradation in a subject with normal lung function. This method comprises measuring, by mass spectrometry, a marker of elastic fiber injury selected from the group consisting of desmosine, isodesmosine, and a combination thereof in a sample from the subject.

### BRIEF DESCRIPTION OF THE DRAWINGS

FIG. 1 shows the chemical synthesis of DES-d4.

FIG. 2 shows a 1H nuclear magnetic resonance (NMR) (D2O, 300 MHz) spectrum of DES-d4: d 8.56 (2H, s, H2/6), 4.52 (2H, t, J=6.9 Hz, H7), 4.14-4.12 (2H, m, H20/20′), 4.05-3.96 (1H, m, H16), 4.05-3.96 (1H, m, H11), 3.08-2.91 (4H, m, H18/18′), 2.24-2.22 (4H, m, H19/19′), 2.10 (2H, m, H15), 2.04-1.97 (4H, m, H8/10), 1.41 (2H, m, H9).

FIG. 3 shows an electrospray ionization (ESI) mass spectrum of DES-d4.

FIG. 4A shows calibration for DES and IDS levels from the peak ratios of (DES+IDS)/IS.

FIG. 4B shows calibration for DES and IDS from the peak ratios of DES/IS or IDS/IS.

FIG. 5 shows three LC-MS/MS chromatograms of DES, IDS, and IS (DES-d4) in body fluids: A) urine, B) plasma, and C) bronchoalveolar lavage fluid (BALF).

FIG. 6 shows total and free DES+IDS levels in plasma and total DES+IDS levels in BALF in COPD patients.

FIG. 7 shows the effect of intravenous alpha-1 antitrypsin augmentation therapy on plasma levels of desmosine and isodesmosine (DI) in alpha-1 antitrypsin deficiency. The upper boundary of each box indicates 75th percentile and the lower boundary indicates 25th percentile. Whiskers (error bars) above and below each box indicate the maximum and minimum. Mean±standard deviation (SD) of normal: 0.22±0.04, n=47; mean±SD of augmentation: 0.25±0.01, n=50; mean±SD of no augmentation: 0.36±0.01, n=50. The results of the t-test are as follows: normal vs. augmentation: P=0.0035; augmentation vs. no augmentation: P<0.0001; and normal vs. no augmentation: P<0.0001.

FIGS. 8A and 8B show the effect of intravenous alpha-1 antitrypsin augmentation therapy on levels of desmosine and isodesmosine (DI) in plasma.

FIG. 9 shows the effect of intravenous alpha-1 antitrypsin augmentation therapy on levels of desmosine and isodesmosine (DI) in epithelial lining fluid obtained by BALF.

FIGS. 10A and 10B show the effect of aerosol alpha-1 antitrypsin augmentation therapy on levels of desmosine and isodesmosine (DI) in plasma.

FIG. 11 shows the effect of aerosol alpha-1 antitrypsin augmentation therapy on levels of desmosine and isodesmosine (DI) in epithelial lining fluid obtained by BALF.

FIG. 12 shows the correlation of DI in plasma and BALF 12 weeks after intravenous augmentation therapy.

FIGS. 13A and 13B show the effect of aerosol alpha-1 antitrypsin augmentation therapy on urinary desmosine and isodesmosine (DI): Ratios of Free DI to Total DI.

FIG. 14 shows the relationship of age to plasma levels of desmosine and isodesmosine (DI) in normal subjects and alpha-1 deficient subjects receiving and not receiving intravenous alpha-1 antitrypsin augmentation therapy.

FIG. 15 shows plasma levels of desmosine and isodesmosine (DI) in early stage alpha-1 antitrypsin deficiency over a 3 year interval.

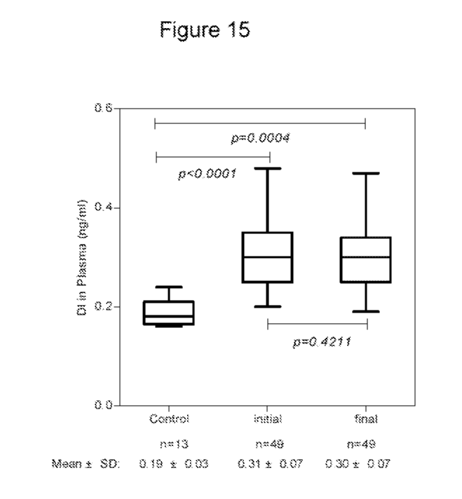

FIG. 16 shows correlations of plasma levels of desmosine and isodesmosine (DI) with age (A), diffusing capacity of the lung for carbon monoxide (DL_{CO+}) (B) and FEV₁ (C).

### DETAILED DESCRIPTION OF THE INVENTION

One embodiment of the present invention is a method for measuring the amount of a marker of elastic fiber injury selected from the group consisting of DES, IDS, and a combination thereof in a sample. This method comprises contacting the sample with a compound of formula (1), also referred to herein as “DES-d₄” or “DES-d₄15”:

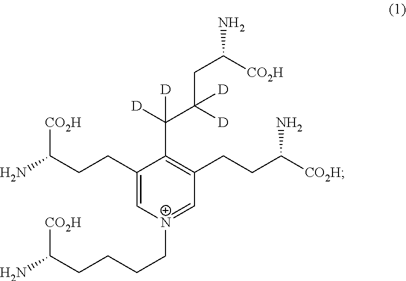 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

and carrying out mass spectrometry on the sample containing the compound of formula (1). In formula (1) of the present invention, “D” means deuterium. The deuterium atoms are added to formula (I) using any convenient method, such as the method disclosed in Example 1 below.

As used herein, the term “elastic fiber injury” means any disruption to elastin-containing components of the skin, blood vessels, lungs, and other tissues that results in degradation or reduced integrity of these elastin-containing components.

As used herein, the term “mass spectrometry” means any technique that identifies the molecular components within a sample by detecting the spectra of mass/charge ratios and relative abundance of the molecular components within the sample. Types of mass spectrometry include, but are not limited to, liquid chromatography mass spectrometry (LC-MS), liquid chromatography tandem mass spectrometry (LC-MS/MS), isotope-dilution liquid chromatography tandem mass spectrometry, accelerator mass spectrometry, gas chromatography mass spectrometry, inductively-coupled plasma mass spectrometry, thermal ionization mass spectrometry, and spark source mass spectrometry. Preferably, in the present invention the mass spectrometry used is LC-MS or LC-MS/MS. More preferably, the mass spectrometry is liquid chromatography tandem mass spectrometry (LC-MS/MS).

As used herein, the term “sample” means any substance obtained from an organism that can be analyzed to determine some trait of the organism or some condition that affects the organism. Examples of samples include, but are not limited to, connective tissue matrices, urine, plasma, serum, sputum, and bronchoalveolar lavage fluid (BALF).

In one aspect of this embodiment, the amount of the compound of formula (1) is pre-determined.

In another aspect of this embodiment, the sample which has been contacted with the compound of formula (1) is subjected to acid hydrolysis prior to mass spectrometry.

As used herein, the term “acid hydrolysis” means the breakdown or degradation of a substance by exposure to an acid, yielding component parts of the substance. In the context of the present invention, acid hydrolysis is used to analyze total DES and IDS in a sample. Some DES and IDS exist freely in samples from patients with a disease characterized by elastic fiber injury, but to measure total DES and IDS levels, crosslinked DES and IDS must first be released from elastin-containing structures by acid hydrolysis.

In an additional aspect of this embodiment, the sample is selected from the group consisting of connective tissue matrices, urine, plasma, serum, sputum, bronchoalveolar lavage fluid (BALF), and combinations thereof. Other samples may be used so long as they can be used in the methods of the present invention.

As used herein, the term “connective tissue matrices” means the extracellular components that provide structural support to an organism. Extracellular matrices include, but are not limited to, interstitial matrix and basement membrane. Components of these matrices include, but are not limited to, fibronectin, collagen, laminin, and elastin.

In another aspect of this embodiment, the sample is obtained from a subject suspected of having a disease characterized by elastic fiber injury.

As used herein, a “subject” is a mammal, preferably, a human. In addition to humans, categories of mammals within the scope of the present invention include, for example, agricultural animals, domestic animals, laboratory animals, etc. Some examples of agricultural animals include cows, pigs, horses, goats, etc. Some examples of domestic animals include dogs, cats, etc. Some examples of laboratory animals include rats, mice, rabbits, guinea pigs, etc.

In the present invention, diseases characterized by elastic fiber injury include, but are not limited to, atherosclerosis, aortic aneurysm, skin lesion, cystic fibrosis, and chronic obstructive pulmonary disease (COPD). Preferably, the disease is COPD. More preferably, the COPD is pulmonary emphysema.

In an additional aspect of this embodiment, the amount of DES in the sample is calibrated in relation to the amount of the compound of formula (1). As used herein, the term “calibrated” means to adjust by comparing to a standard, e.g., a known quantity of the compound of formula (1). Methods of calibrating are as set forth herein. In another aspect of this embodiment, the amount of DES and IDS in the sample is calibrated in relation to the amount of the compound of formula (1).

Another embodiment of the present invention is a method for diagnosing whether a subject has a disease characterized by an elastic fiber injury. This method comprises:

(a) contacting a compound of formula (1):

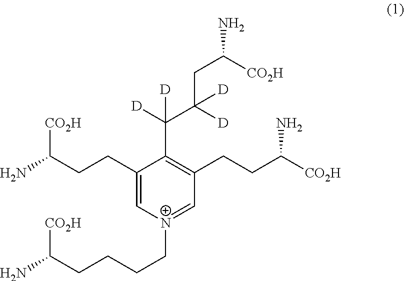 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

with a sample obtained from the subject; and

(b) measuring, by mass spectrometry, the amount of a marker of elastic fiber injury selected from the group consisting of DES, IDS, and a combination thereof in the sample.

As used herein, the terms “diagnose,” “diagnosing,” and grammatical variations thereof mean identifying, e.g., a disease. The diseases characterized by elastic fiber injury, suitable and preferred subject(s), as well as various types of mass spectrometry according to the present invention are as disclosed above.

In an additional aspect of this embodiment, the amount of DES in the sample is calibrated in relation to the amount of the compound of formula (1). In another aspect of this embodiment, the amount of DES and IDS in the sample is calibrated in relation to the amount of the compound of formula (1).

An additional embodiment of the present invention is a method for improving the accuracy and precision of mass spectroscopy analysis of a marker of elastic fiber injury in a sample, the marker being selected from the group consisting of DES, IDS, and a combination thereof. This method comprises:

(a) contacting a compound of formula (1):

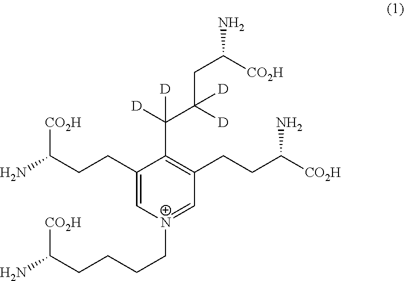 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

with a sample from a subject suspected of having a disease characterized by elastic fiber injury;

(b) carrying out acid hydrolysis of the sample from step (a) containing the compound of formula (1); and

(c) carrying out mass spectrometry on the acid hydrolyzed sample from step (b).

As used herein, the terms “accuracy” and “precision” mean the degree to which the analysis yields the true concentration of a compound in a sample and the degree to which subsequent analyses yield consistent results, respectively.

The diseases characterized by elastic fiber injury, suitable and preferred subject(s), as well as various types of mass spectrometry according to the present invention are as disclosed above.

In an additional aspect of this embodiment, the amount of DES in the sample is calibrated in relation to the amount of the compound of formula (1). In another aspect of this embodiment, the amount of DES and IDS in the sample is calibrated in relation to the amount of the compound of formula (1).

Another embodiment of the present invention is a kit for determining, by mass spectrometry, the amount of a marker of elastic fiber injury in a sample from a subject. This kit comprises a compound of formula (1):

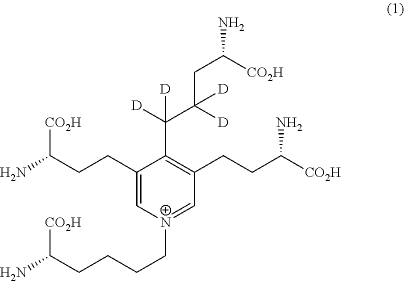 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

and instructions for use thereof, wherein the marker of elastic fiber injury is selected from the group consisting of DES, IDS, and a combination thereof. The kit may optionally contain one or more containers for, e.g., the IS and/or various reagents, including buffer solutions and reagents for acid hydrolysis, etc., for carrying out the methods disclosed herein. The containers may be made of any appropriate material including glass, plastic, etc. The IS and/or other various reagents may be present in the kit in any convenient form, e.g., as powders, as lyophilized forms and/or in liquid forms.

The diseases characterized by elastic fiber injury, suitable and preferred subject(s), as well as various types of mass spectrometry are as disclosed above.

Another embodiment of the present invention is a method for preventing the progression of the effects associated with alpha-1 antitrypsin deficiency (AATD) in a subject with normal lung function. This method comprises

(a) measuring, by mass spectrometry, a marker of elastic fiber injury selected from the group consisting of desmosine, isodesmosine, and a combination thereof in a sample from the subject; and

(b) administering AATD augmentation therapy if the subject has a higher than normal amount of the marker of elastic fiber injury.

As used herein, “normal lung function” means that the subject's pulmonary system is functioning in a typical fashion, e.g., as judged by a medical professional using traditional tests, such as total lung capacity.

As used herein, “a normal amount” of the marker of elastic fiber injury means that the desmosine levels and/or isodesmosine levels are at or below the average for a population. For example, as disclosed herein, the total desmosine and isodesmosine levels in plasma is about 0.19±0.02 ng/ml for normal subjects who are non-smokers and who are not exposed to second-hand smoke.

Suitable and preferred subjects, samples, and methods for obtaining the samples are as disclosed above.

In one aspect of this embodiment, the sample is contacted with a compound of formula (1):

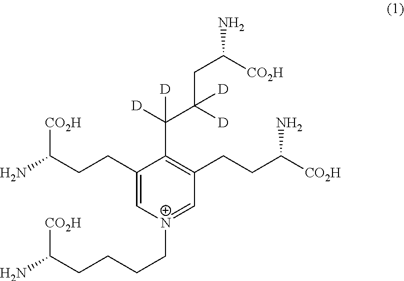 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

prior to carrying out mass spectroscopy.

In an additional aspect of this embodiment, the amount of DES in the sample is calibrated in relation to the amount of the compound of formula (1). In another aspect of this embodiment, the amount of DES and IDS in the sample is calibrated in relation to the amount of the compound of formula (1).

An additional embodiment of the present invention is a method for detecting lung elastin degradation in a subject with normal lung function. This method comprises measuring, by mass spectrometry, a marker of elastic fiber injury selected from the group consisting of desmosine, isodesmosine, and a combination thereof in a sample from the subject.

Suitable and preferred subjects, samples, and methods for obtaining the samples are as disclosed above.

In one aspect of this embodiment, the sample is contacted with a compound of formula (1):

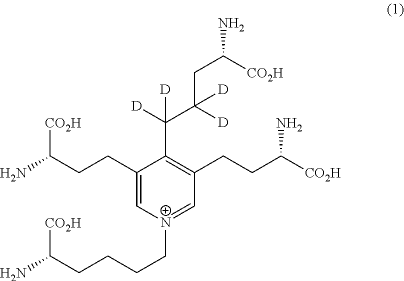 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]

prior to carrying out mass spectroscopy.

In an additional aspect of this embodiment, the amount of DES in the sample is calibrated in relation to the amount of the compound of formula (1). In another aspect of this embodiment, the amount of DES and IDS in the sample is calibrated in relation to the amount of the compound of formula (1).

The following examples are provided to further illustrate the methods of the present invention. These examples are illustrative only and are not intended to limit the scope of the invention in any way.

### EXAMPLES

### Example 1

### Materials and Methods

### Reagents

For the chemical synthesis of DES-d₄: deuterium gas (99.9% atom %) was purchased from Sigma (St. Louis, Mo.). All reactions were conducted with magnetic stirring using dry solvents unless otherwise indicated. CD₃OD for the reactions was purchased from Kanto Chemicals (Tokyo, Japan). All reagents were obtained from commercial suppliers and used without further purification unless otherwise stated. Analytical thin layer chromatography (TLC) was performed on Silica gel 60 F254 plates produced by Merck (Whitehouse Station, N.J.). ¹H NMR spectra were recorded on a JEOL JNM-EXC 300 spectrometer (300 MHz) (JEOL Ltd., Tokyo, Japan). ¹H NMR data are reported as follows: chemical shift (5, ppm), integration, multiplicity (s, singlet; d, doublet; t, triplet; q, quartet; m, multiplet), coupling constants (J) in Hz. ESI-MS spectra were recorded on a JEOL JMS-T100LC instrument (JEOL Ltd., Tokyo Japan). For the LC-MS/MS analysis: DES and IDS standards (mixed 50% DES and 50% IDS) were purchased from the Elastin Products Company (Owensville, Mich.), CF1 cellulose powders were purchased from Whatman (Clifton, N.J.), and all other reagents were obtained from Sigma (St. Louis, Mo.).

### Chemical Synthesis of DES-d₄

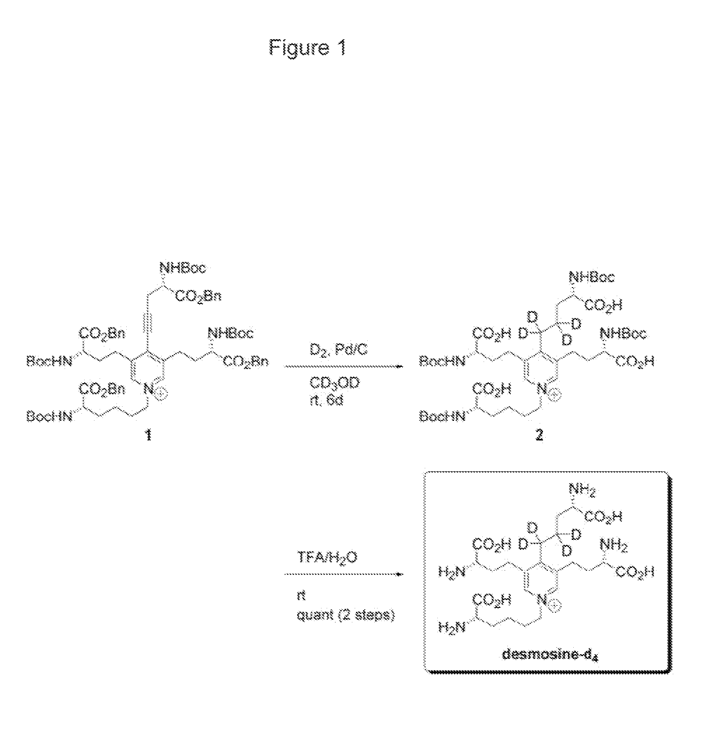

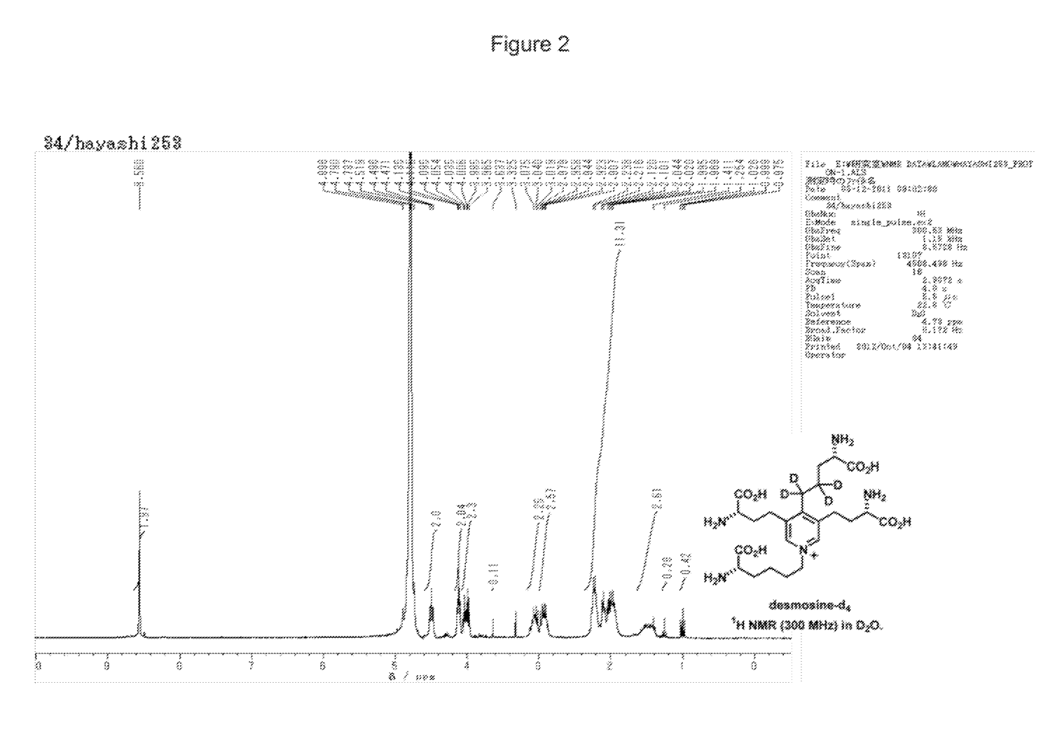

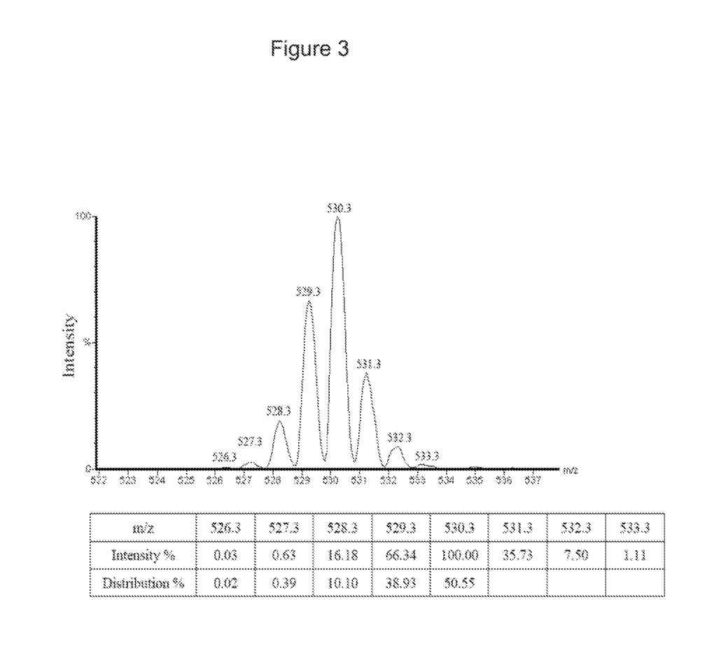

The starting material is the 4-alkynyl DES derivative compound 1 (FIG. 1) which was previously synthesized (Usuki, T. et al., 2012, Yanuma, H. et al., 2012). A solution of compound 1 (35.2 mg, 27.4 μmol, 1.0 eq) in CD₃OD (0.9 mL) was treated with 10% Pd/C (145.6 mg, 0.14 mmol, 5.0 eq) and, under D₂ atmosphere, deuterated using a balloon at room temperature. After stirring for 6 days at room temperature, the reaction mixture was separated by filtration through a Celite pad on neutral silica gel eluting with MeOH and the filtrate was then concentrated in vacuo to afford a crude mixture of compound 2 as a yellow solid; ESI-MS (m/z) calculated for C₄₄D₄H₆₈N₅O₆ [M]⁺: 930.52. found: 930.39. The obtained product was used in the next reaction without further purification. A mixture of TFA and distilled water (7.0 mL, TFA/water=95/5) was added to the crude 2 at room temperature and stirred for 2 hours. The solvent was removed in vacuo. Purification on C18 column chromatography (0.1% TFA in distilled water) afforded the desired DES-d₄ as a yellow solid (28.9 mg, 44.9 μmol, quant (2 steps)); R_{f} 0.22 [MeOH (0.1% TFA)/H₂0 (0.1% TFA)=1:9]. The structure of DES-d₄ was confirmed by both NMR (FIG. 2) and mass spectra (FIG. 3). Newly synthesized DES-d₄ possesses all four deuterium atoms at the stable alkane carbons which are stable toward acidic conditions. The synthesized deutereo-DES consists of four deutereo-isotopomers: DES-d₄ (50.53%), -d₃ (38.93%), -d₂ (10.10%), and -d₁ (0.39%) as determined by the MS spectra shown in FIG. 3. The most abundant DES-d₄ ion (m/z 530) is used for the isotope-dilution LC-MS/MS analysis.

### Biological Samples for DES and IDS Analysis

Ten plasma and 16 BALF samples from well characterized COPD patients were obtained from the FORTE study (Roth, M. D. et al., 2006).

### Isotope Dilution LC-MS/MS Analysis of DES and IDS

LC-MS/MS analysis was performed by modification of the standardized three step procedure published previously (Ma, S. et al., 2011). The procedure for the analysis of a fluid sample is as follows.

Step 1: Acid hydrolysis: DES-d₄ IS (5 ng) was added to the analytical samples (0.5 ml) in a glass vial with equal volumes of concentrated HCl (37%). Air in the vial was displaced with nitrogen, and was heated at 110° C. for 24 hours. The hydrolyzed sample was filtered and evaporated to dryness. For analysis of the free (unconjugated) forms of DES and IDS, the samples were analyzed directly without the HCl hydrolysis.

Step 2: Cellulose (CF1) cartridge extraction: The acid hydrolyzed samples (after drying under vacuum or nitrogen stream to remove residual acid) or unhydrolyzed samples (for free DES/IDS analysis) were dissolved in 2 ml of n-butanol/acetic acid/water (4:1:1), and applied onto a 3 ml cellulose cartridge, which was prepared by introduction of 3 ml of 5% CF1 cellulose powder slurry in n-butanol/acetic acid/water (4:1:1). The cellulose powder slurry must be a well dispersed slurry, necessitating stirring for 24 hours. The cartridge was washed 3 times with 3 ml of n-butanol/acetic acid/water (4:1:1), and the samples retained in the cartridge were eluted with 3 ml of water, dried and dissolved in 100 μl of LC mobile phase and analyzed by LC-MS/MS.

Step 3: LC-MS/MS analysis: A TSQ Discovery electrospray tandem mass spectrometer (Thermo Fisher Scientific) was used for LC-MS/MS analysis. HPLC conditions used were a 2 mm×150 mm dC18 (3 μm) column (Waters, Mass.) and the mobile phase A (7 mM HFBA/5 mM NH₄Ac in water) and B (7 mM HFBA/5 mM NH₄Ac in 80% acetonitrile) were programmed linearly from 100% A to 82% A in 12 m. Quantitation was performed by selected reaction monitoring (SRM) of the transitions of both DES and IDS (m/z 526 to m/z 481+m/z 397) and the IS (m/z 530 to m/z 485), with collision energy set at 33 V for both transitions, collision gas pressure was 1.5 mTorr, tube lens at 132 V, with sheath gas pressure set at 45 and auxiliary gas pressure at 6 units and ion spray voltage at 3.8 kV. The scan time was set at 1.00 ms and both quadrupoles (Q1 and Q3) were at 0.7 Da FWHM.

### Statistical Analysis

A t-test adjusted for unequal variance was used to test the null hypothesis. The level of significance was 0.05. The p-values were calculated based on the summed values of DES and IDS using the unpaired t-test (all calculations were performed using GraphPad Prism 4.2).

### Example 2

### Stability of DES-d₄ as the IS

The stability of DES-d₄ and reliability during use as the IS for the isotope dilution LC-MS/MS analysis of DES and IDS were examined. DES-d₄ (1000 ng) was added to DES and IDS in five concentrations of 50, 100, 150, 200, and 1000 ng/ml in 6N HCl and the mixed solutions were heated at 110° C. for 24, 48, and 72 hours. The mass spectral analysis of the resulting solution showed DES-d₄ to be stable with nearly complete recovery (Table 1).

TABLE 1
Stability of DES-d₄ (IS) in 6N HCl at 110° C.

| DES-d4 | | | | | | | | | | | | | | | |
|---|---|---|---|---|---|---|---|---|---|---|---|---|---|---|---|
| Recovery | Con. 1 | | | Con. 2 | | | Con. 3 | | | Con. 4 | | | Con. 5 | | |
| % | 24 hr | 48 hr | 72 hr | 24 hr | 48 hr | 72 hr | 24 hr | 48 hr | 72 hr | 24 hr | 48 hr | 72 hr | 24 hr | 48 hr | 72 hr |
| Average | 101.28 | 107.52 | 98.08 | 112.20 | 110.78 | 109.96 | 109.93 | 105.03 | 101.03 | 101.22 | 91.01 | 83.48 | 98.20 | 98.22 | 99.41 |
| (n = 3) | | | | | | | | | | | | | | | |
| SD | 4.71 | 10.72 | 9.30 | 9.37 | 6.45 | 8.27 | 4.45 | 5.24 | 7.03 | 6.00 | 3.76 | 7.82 | 7.18 | 4.12 | 6.83 |
| % CV | 4.65 | 9.97 | 9.48 | 8.35 | 5.82 | 7.52 | 4.05 | 4.99 | 6.96 | 5.93 | 4.13 | 9.37 | 7.31 | 4.19 | 6.87 |

* The mixtures of DES + IDS and the IS (DES-d₄) of the five concentrations below are hydrolyzed in 6N HCl for 24 hr, 48 hr, and 72 hr and the recoveries are analyzed by LC-MS/MS: Con. 1: DES + IDS 100 ng, IS 1000 ng; Con. 2: DES + IDS 200 ng, IS 1000 ng; Con. 3: DES + IDS 300 ng, IS 1000 ng; Con. 4: DES + IDS 400 ng, IS 1000 ng; Con. 5: DES + IDS 500 ng, IS 1000 ng (all in 1 ml of 6N HCl)

DES and IDS have been shown to be stable during acid hydrolysis in 6N HCl (Ma, S. et al., 2011). We further confirm recovery of DES and IDS substrates in the presence of the IS in the five different concentrations, which are the most likely concentrations to be used in the analysis of biological samples. The results, as shown in Table 2, give consistent ratios of DES+IDS to the IS in all five concentrations. These results demonstrate that DES-d₄ can serve as a reliable IS for measuring DES and IDS under acid hydrolysis.

TABLE 2
Precision of the ratios of DES + IDS/IS in 6N HCl at 110° C.

| (DES + IDS)/IS | Con. 1 | | | Con. 2 | | | Con. 3 | | | Con. 4 | | | Con. 5 | | |
|---|---|---|---|---|---|---|---|---|---|---|---|---|---|---|---|
| Recovery % | 24 hr | 48 hr | 72 hr | 24 hr | 48 hr | 72 hr | 24 hr | 48 hr | 72 hr | 24 hr | 48 hr | 72 hr | 24 hr | 48 hr | 72 hr |
| Average (n = 3) | 91.32 | 93.85 | 91.02 | 91.85 | 90.71 | 92.75 | 99.95 | 100.05 | 101.69 | 100.07 | 103.06 | 103.34 | 95.65 | 101.12 | 100.03 |
| SD | 4.59 | 8.57 | 4.65 | 2.59 | 4.51 | 4.11 | 5.44 | 4.67 | 7.49 | 1.57 | 4.23 | 4.67 | 8.71 | 3.78 | 4.66 |
| % CV | 5.02 | 9.13 | 5.11 | 2.81 | 4.97 | 4.43 | 5.45 | 4.66 | 7.36 | 1.56 | 4.10 | 4.52 | 9.11 | 3.74 | 4.66 |

* The mixtures of DES + IDS and the IS (DES-d₄) of the five concentrations below are hydrolyzed in 6N HCl for 24 hr, 48 hr, and 72 hr and the recoveries are analyzed by LC-MS/MS: Con. 1: DES + IDS 100 ng, IS 1000 ng; Con. 2: DES + IDS 200 ng, IS 1000 ng; Con. 3: DES + IDS 300 ng, IS 1000 ng; Con. 4: DES + IDS 400 ng, IS 1000 ng; Con. 5: DES + IDS 500 ng, IS 1000 ng (all in 1 ml of 6N HCl).

### Example 3

### Isotope-Dilution LC-MS/MS Analysis of DES and IDS

The synthesized DES-d₄ was used as the IS to develop a new isotope-dilution LC-MS/MS analysis of DES and IDS. The reproducibility and the accuracy of DES and IDS measurements were studied both by their quantitative linearity and their recovery from the biological matrix.

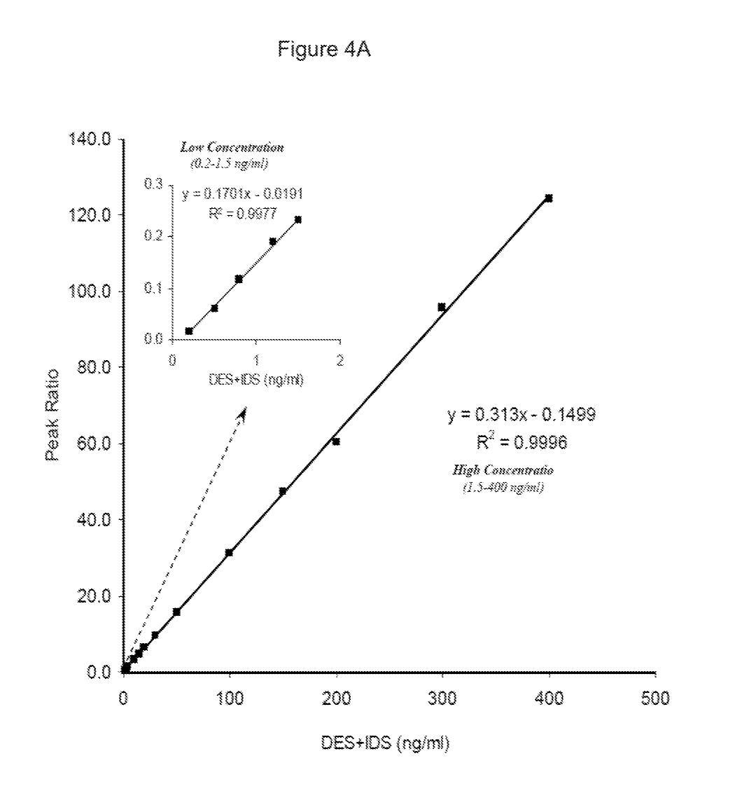

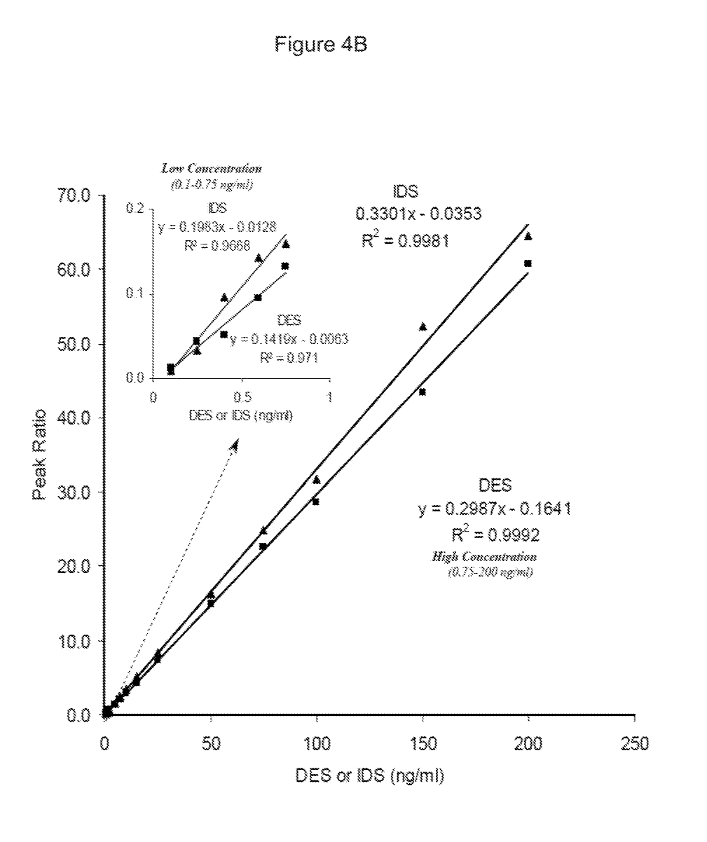

The calibration curves for DES and IDS quantifications were prepared with 15 dilutions from 0.05 to 400 ng/ml in the presence of 1 μg of IS (DES-d4). Excellent linearity of the isotope ratios, DES+IDES to IS (FIG. 4A) or individual DES or IDS to IS (FIG. 4B) were obtained. Good inter-assay precision (CV %) and accuracy (% bias) of the calibrations were achieved for the DES+IDS levels from 2.0 ng/ml up to 400 ng/ml. At a lower concentration of DES+IDS from 0.2-1.5 ng/ml (insert in FIG. 4A), less inter-assay precision could be observed due to the insufficient ion stability at a lower ion population in the ESI-MS spectrometer. We believe precision at such low concentrations can be achieved by the improved ion stability when using a newer model of ESI-MS instrument.

The isotope-dilution analysis of DES or IDS can also be achieved by the use of the individual calibration as shown in FIG. 4B, but it should be mentioned that a slightly higher imprecision could be observed with the individual DES and IDS measurements due to an incomplete base-peak chromatographic separation of the two isomers. This can be improved by greater chromatographic separation when such individual DES and IDS measurements are required.

### Example 4

### The Recovery of DES/IDS from Connective Tissue Matrices

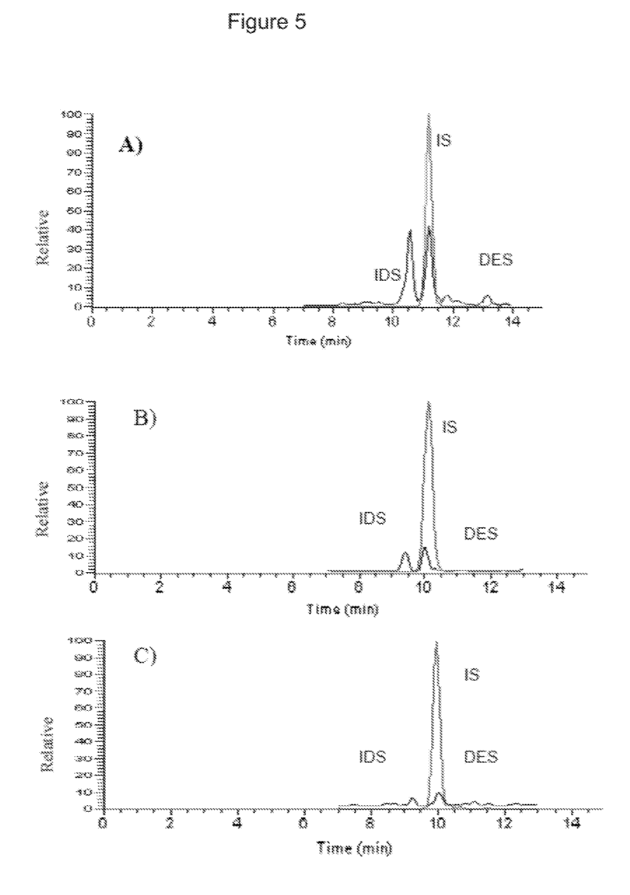

FIG. 5 shows a typical LC-MS/MS chromatogram of the isotope-dilution analysis of three representative connective tissue matrices: urine, plasma, and BALF. The recovery of DES and IDS from the quality control samples of two tissue matrices, urine and plasma, are shown in Table 3. These results demonstrate that the total DES+IDS and the free DES+IDS in plasma and urine samples can be measured by the isotope-dilution LC-MS/MS with great accuracy.

TABLE 3
Recovery of DES and IDS in biological matrices
A) Plasma (Total DES + IDS)

| | | | LOW QC | MID QC | HIGH QC |
|---|---|---|---|---|---|
| Plasma | | | Endogenous | Endogenous | Endogenous |
| (Total | | | Spike | Spike | Spike |
| DES + IDS) | | Endogenous | 0.1 ng/m1 | 0.5 ng/m1 | 1.1 ng/m1 |
| DES + IDS | Average (n = 3) | 0.55 | 0.66 | 1.07 | 1.68 |
| | (ng/ml) | | | | |
| | SD | 0.11 | 0.13 | 0.08 | 0.01 |
| Precision | % CV | 6.88 | 7.54 | 3.79 | 0.22 |
| Accuracy | % bias | | 1.31 | 4.82 | 5.43 |

B) Plasma (Free DES + IDS)

| | | | LOW QC | MID QC | HIGH QC |
|---|---|---|---|---|---|
| | | | Endogenous | Endogenous | Endogenous |
| Plasma | | Endogenous | (free-DES + | (free-DES + | (free-DES + |
| (Free | | (free DES + | IDS) Spike | IDS) Spike | IDS) Spike |
| DES + IDS) | | IDS) | 0.3 ng/m1 | 0.9 ng/m1 | 1.8 g/m1 |
| DES + IDS | Average (n = 3) | 0.09 | 0.39 | 1.00 | 1.90 |
| | (ng/ml) | | | | |
| | SD | 0.05 | 0.10 | 0.02 | 0.14 |
| Precision | % CV | 5.25 | 8.29 | 1.33 | 5.16 |
| Accuracy | % bias | | 0.27 | 5.17 | 5.40 |

C) Urine (Total DES + IDS)

| | | | LOW QC | MID QC | HIGH QC |
|---|---|---|---|---|---|
| Urine | | | Endogenous | Endogenous | Endogenous |
| (Total | | | Spike | Spike | Spike |
| DES + IDS) | | Endogenous | l ng/ml | l0 ng/m1 | 30 ng/m1 |
| DES + IDS | Average (n = 3) | 12.70 | 13.44 | 23.68 | 42.04 |
| | (ng/ml) | | | | |
| | SD | 1.03 | 0.68 | 2.21 | 0.57 |
| Precision | % CV | 8.15 | 5.03 | 9.32 | 1.37 |
| Accuracy | % bias | | −2.09 | 7.71 | −5.25 |

D) Urine (Free DES + IDS)

| | | | LOW QC | MID QC | HIGH QC |
|---|---|---|---|---|---|
| | | | Endogenous | Endogenous | Endogenous |
| Urine | | Endogenous | (free-DES + | (free-DES + | (free-DES + |
| (Free | | (free-DES + | IDS) Spike | IDS) Spike | IDS) Spike |
| DES + IDS) | | IDS) | l ng/ml | l0 ng/m1 | 30 ng/m1 |
| DES + IDS | Average (n = 3) | 6.45 | 7.62 | 16.50 | 36.36 |
| | (ng/ml) | | | | |
| | SD | 1.00 | 1.02 | 1.47 | 2.69 |
| Precision | % CV | 7.77 | 7.14 | 6.40 | 6.31 |
| Accuracy | % bias | | 2.66 | 0.87 | −1.39 |

* The standard urine samples (plasma 0.5 ml and urine 1.0 ml) were spiked with DES and IDS in the three concentrations as described in the Tables. DES + IDS levels were determined by the isotope dilution LC-MS/MS analysis.

### Example 5

### Measurements of DES and IDS as Biomarkers of Elastin Degradation in COPD

The developed isotope dilution LC-MS/MS analysis was used to examine DES and IDS levels in 10 plasma and 16 BALF samples from well characterized patients with moderate to severe COPD who were entered into the FORTE study (Roth, M. D. et al., 2006). A typical example of the LC-MS/MS chromatogram in plasma and BALF is shown in FIG. 5; where (A) represents total DES and IDS levels in plasma after acid hydrolysis, (B) represents free DES and IDS levels (without acid hydrolysis) in plasma, and (C) represents total DES and IDS levels in BALF.

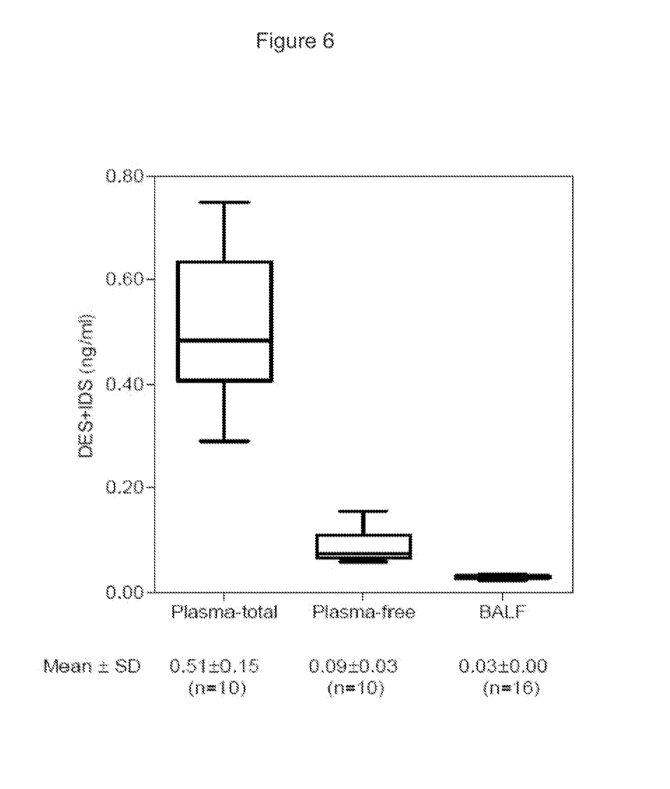

The DES+IDS levels in these COPD patients were measured by the above described isotope-dilution LC-MS/MS analysis and results are summarized in FIG. 6. The levels of total DES+IDS in plasma (after acid hydrolysis of plasma) shows an average 0.51±0.15, which is in the significantly elevated ranges of COPD patients previously reported, where the total DES+IDS levels were 0.19±0.02 for normal and 0.65±0.14 ng/ml for COPD patients (Ma et al., 2007). In addition, the increased sensitivity and specificity of this newly developed isotope-dilution LC-MS/MS method enable measurement of levels of free DES and IDS in plasma 0.09±0.03 ng/ml and total DES and IDS in BALF 0.03±0.01 ng/ml.

### Example 6

We have chemically synthesized a stable deuterated isotope, DES-d₄, that possesses all four deuterium atoms at the alkanyl carbons of the alkyl amino acid substitution in the DES molecule and is stable toward acid hydrolysis. The latter trait is required in the measurement of the two crosslinking molecules, DES and IDS, as biomarkers of elastic tissue degradation. Our recent achievement in the total synthesis of the DES molecule (Usuki, T. et al., 2012, Yanuma, H. et al., 2012), enabled us to synthesize the stable DES-d₄ that can serve as the ideal IS for an accurate isotope-dilution mass spectrometric analysis. Previously published isotope-dilution LC-MS/MS analyses of DES and IDS use a catalytically exchanged deuterium compound obtained from the natural DES as the IS (Boutin, M. et al., 2009 (1), Albarbarawi, O. et al., 2010, Boutin, M. et al., 2009 (2), Lindberg, C. A. et al., 2012). This catalytically exchanged deuterium compound is not stable in acidic conditions and can lead to inaccurate measurements of elastin degradation. The isotope dilution LC-MS/MS using DES-d₄ can be used in the analysis of the crosslinking DES and IDS molecules both under acidic and enzymatic degradations and serves as a generalized method for precise measurements of DES and IDS as biomarkers in biomedical and pathogenic studies involving elastin degradation. This method improves both sensitivity and specificity as shown by the detection of low levels of free DES and IDS in plasma and even lower levels of total DES and IDS in BALF.

Elastin degradation in the lung in COPD is well recognized. COPD is a major health problem worldwide and is now the third leading cause of death in the US (Mannino, D. M. et al., 2007, Rabe, K. F. et al., 2007, Minino, A. M. et al., 2010). We lack therapies which can halt progression of the disease and improve survival. New drug discovery can be aided by the development of biomarkers which can act as indicators of the severity of the disease and responses to therapy. Elastin degradation products can fulfill the need for such a biomarker (Rennard, S. et al., 2012). Urinary DES and IDS have been measured in the past as indicators of elastin degradation, but we believe the measurement of lower levels of DES and IDS in circulatory body fluids such as plasma, serum, or BALF will more directly reflect pathologic or biochemical changes in elastin degradation. Detection of higher than normal levels of free DES and IDS in urine of COPD patients has been reported as a significant marker for elastin degradation in COPD patients (Ma, S. et al., 2003, Ma, S. et al., 2007). However, the detection of free DES and IDS in plasma has not been reported due to its low concentration. Detection of DES and IDS in BALF indicates elastin degradation occurring in the lungs of COPD patients.

The above described isotope-dilution LC-MS/MS analysis can serve as a generalized method for the measurement of DES and IDS as biomarkers in biomedical and pathogenic studies involving elastin degradation.

### Example 7

### Body Fluids for Analysis

In this Example, the plasma samples from patients as well as relatives of patients analyzed in this study were obtained from the Alpha-1 Foundation DNA and Tissue Bank. The Alpha-1 Foundation DNA and Tissue Bank project is sponsored by the Alpha-1 Foundation and is located at the University of Florida College of Medicine in Gainesville Fla. The Alpha-1 Foundation IRB approved this protocol (659-2002). The bronchoalveolar lavage fluid (BALF) and urine samples analyzed in this study were obtained from a previous IRB approved Lung Research Data and Tissue Bank Registry study (UF-IRB577-2002). All subjects signed an informed consent form. Alpha-1 antitrypsin phenotyping and genotyping was performed at the Alpha-1 Genetic Laboratory at the University of Florida.

There were 4 sources of body fluid samples analyzed: 1) plasma samples from 47 patients receiving alpha-1 antitrypsin protein from human blood sources, commercially available, compared with 50 AATD patients not on AAT augmentation and 50 normal subjects; 2) 11 patients with homozygous severe AATD who provided plasma samples before the start of IV therapy and then 12 and 24 weeks later; 3) 10 patients with homozygous AATD receiving IV replacement provided BALF at 12 weeks (the dose administered was 60 mg/kg body weight each week in the above patient groups); and 4) patients received a recombinantly produced AAT which was administered as an aerosol for 8 weeks. (Spencer et al., 2005) (the administered dose was 250 mg of aerosolized transgenic AAT each day). Eight patients provided BALF, 12 patients provided plasma samples, and 5 patients provided urine samples at baseline and after 8 weeks of aerosol therapy.

The following 3 methods were used to define the alpha-1 genomes: 1) genotype by allelic discrimination using TaqMan (S&Z alleles), ABI 7500 Fast Real-time PCR System; 2) AAT Level by Immunogenic assay, Dade Behring Nephelometer BN II and 3) phenotyping by isoelectric focusing, Pharmacia Biotech Multiphore II. The method of analysis for desmosine and isodesmosine (DI) in plasma, BALF and urine is as described. (Ma et al., 2011) The content of DI in BALF was calculated as the concentration of DI per ml of BALF using urea as a marker of dilution. (Rennard et al., 1986).

### Analytic Method for DI

High performance liquid chromatography and tandem mass spectrometry as described (Ma et al., 2011) were used.

### Statistical Methods

The T-test was used to compare the effects of augmentation therapy on the large patient cohorts. The paired T-test was used to compare the changes before and after therapy in individuals. The linear regression was used to determine the relationships of age to levels of DI.

### Results

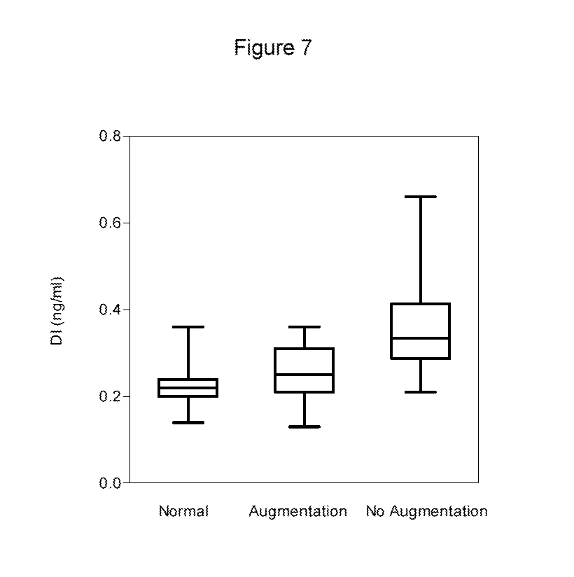

The mean levels of DI in plasma of normal individuals was 0.22 ng/ml and a standard deviation (SD) of 0.04 (n=47), which is compared with a mean of 0.25 ng/ml and a SD of 0.01 (n=50) in patients receiving augmentation therapy p=0.0035 for the difference (n=50). Levels of DI in AATD patients not receiving augmentation (n=50) averaged 0.36 ng/ml with an SD of 0.01, p<0.0001 for comparison of levels of DI in the AATD patients not receiving AAT therapy and the normal controls and between AATD subjects receiving and not receiving augmentation (FIG. 7).

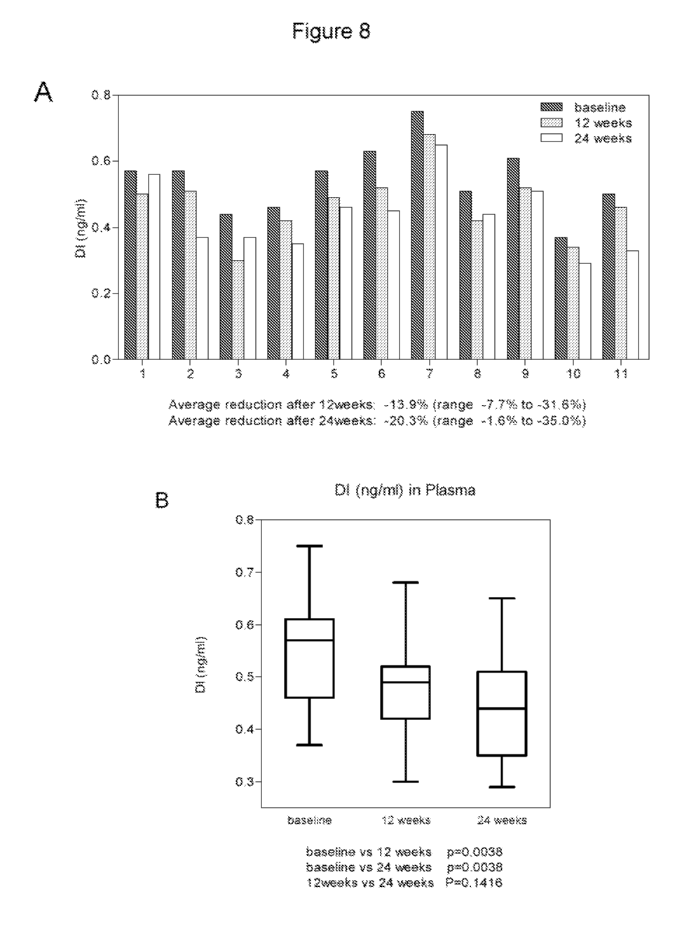

In 11 patients receiving intravenous replacement, levels of DI at baseline and at 12 and 24 weeks of therapy showed statistically significant reductions in DI in plasma at both time points (i.e. −13.9% and −20.3%, p=0.038) (FIGS. 8A and 8B).

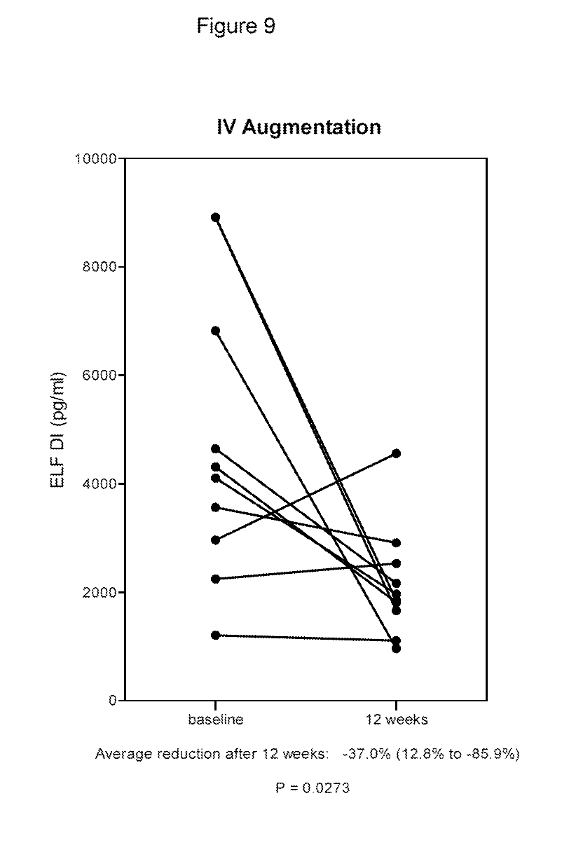

Levels of DI in BALF before and 12 weeks after receiving IV augmentation therapy were analyzed in 10 patients. Levels of DI were reduced in 8 patients and increased in 2. The overall average change was −37% (range: −12.8% to −85.9%) p=0.0273 (FIG. 9).

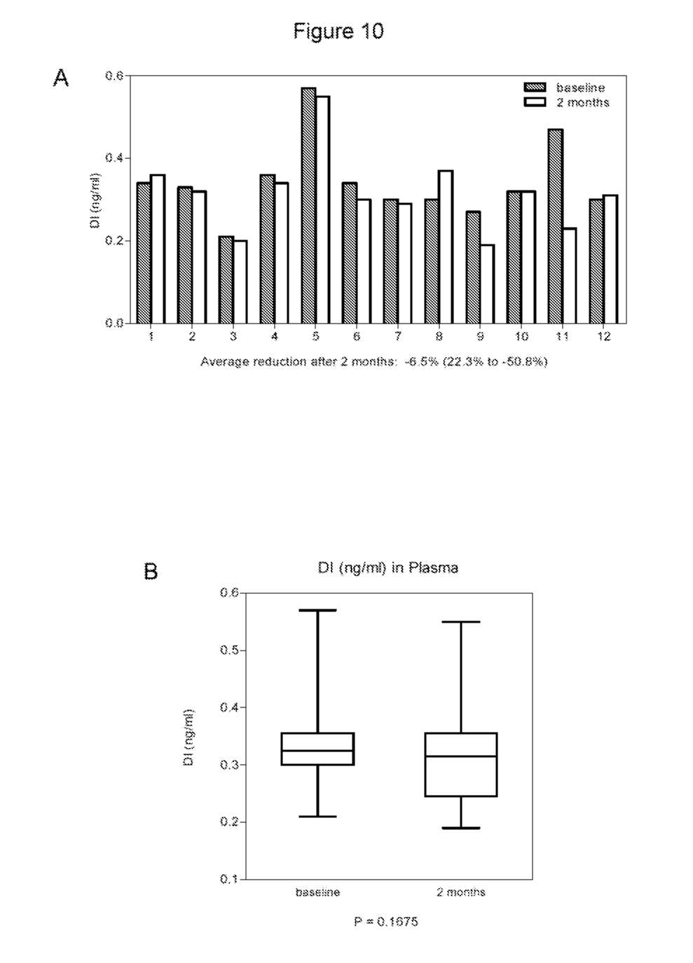

In 12 patients, comparison of before and after aerosol augmentation therapy measurements of DI in plasma showed a mean decrease of 6.5% and a range of percent change from 22.3% to −50.8% from base line. This change in concentration of DI in 12 patients was not statistically significant (p=0.1675) (FIGS. 10A and 10B). The reduction in DI levels in 9 of the 12 patients is statistically significant, an average decrease of 12.1% (range: −0.2% to −50.8%) p=0.0142. The mean increase in DI in 3 patients was 4.3%, which is not statistically significant.

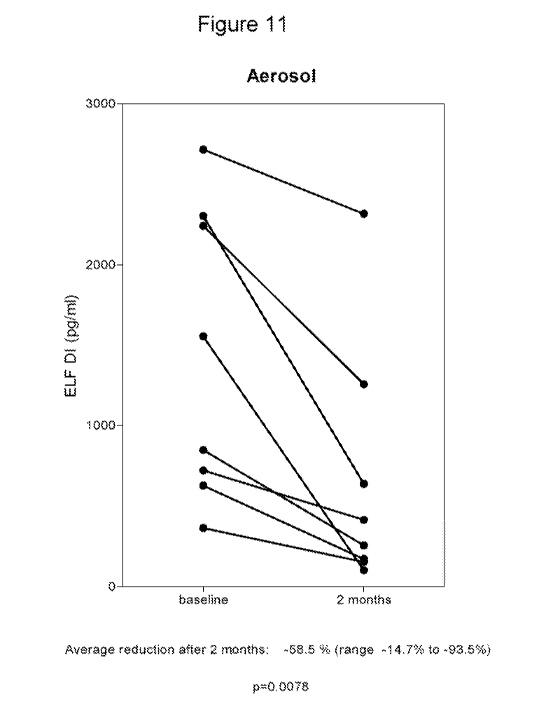

In 8 patients receiving AAT by aerosol administration, DI levels in BALF were all reduced. Mean reduction was −58.5% (range: −14.7% to −93.5%) p=0.0078 (FIG. 11). Analysis of DI in BALF was also done using the total protein content of BALF as a correction factor and the results were similar to that shown.

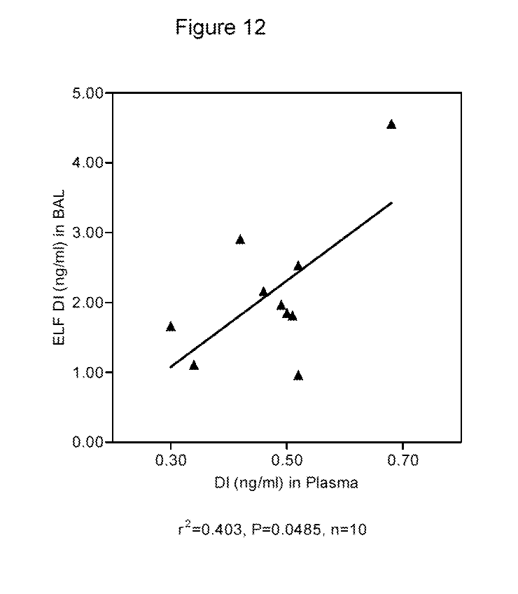

In 10 patients who were receiving intravenous augmentation it was possible to compare levels of DI in bronchoalveolar lavage fluid (BALF) and plasma in the same patients at the same time. There is a positive and significant correlation between the two (FIG. 12).

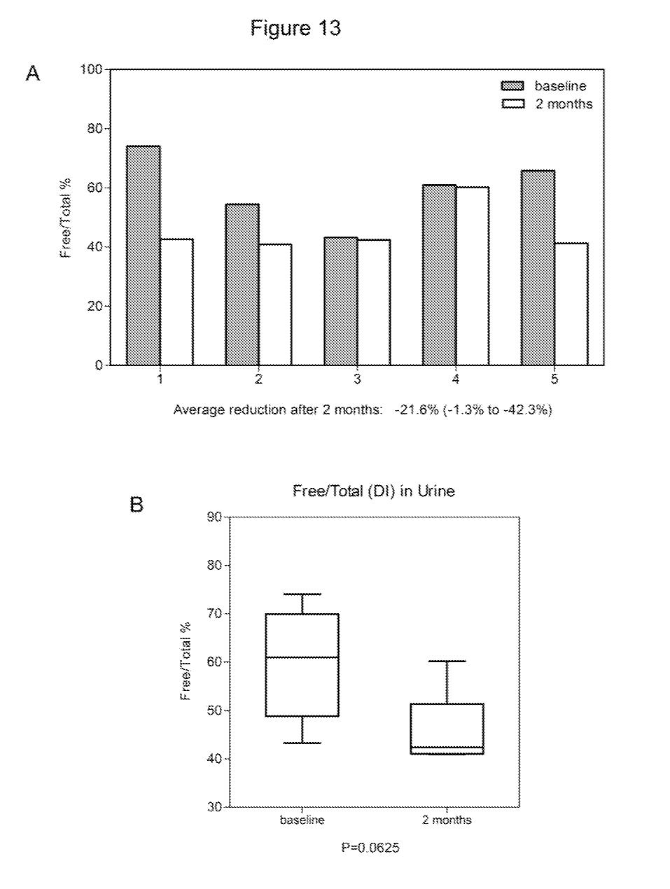

5 subjects' urine samples were obtained before and after administration of augmentation therapy. In 4 of the 5 subjects, there was a reduction in the free component of DI excretion with a range of 0.1-13.0%. One subject showed an increase in excretion of 75.3%. Total excretion of DI was increased post therapy in 3 subjects and decreased in 2. The percent of free DI over total DI excretion was reduced in all 5 subjects with an average reduction of 21.6% and a range of −1.33% to −42.36%. This result was slightly below statistical significance (p=0.0625) (FIGS. 13A and 13B).

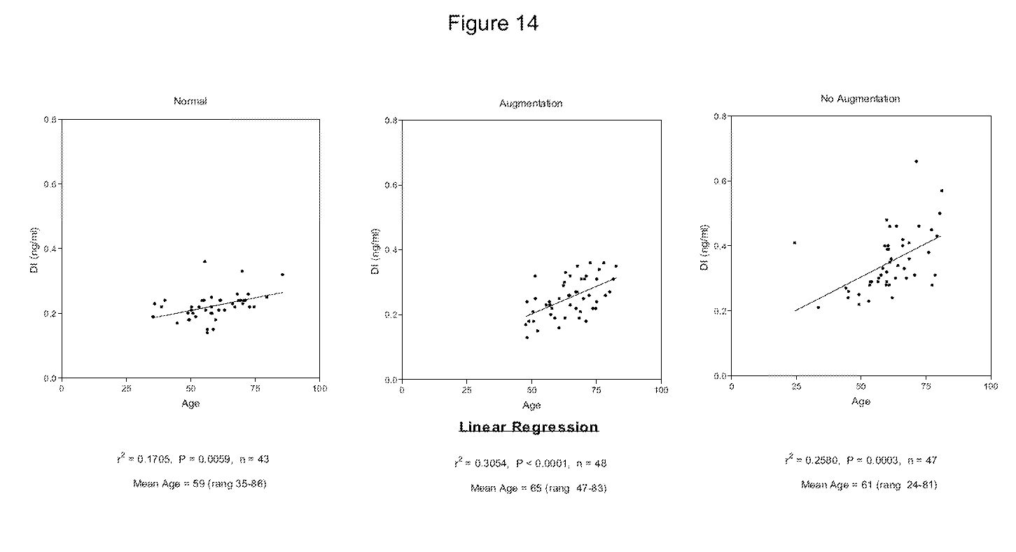

As shown in FIG. 14, there is a statistically significant positive correlation of plasma levels of DI with advancing age in normal subjects, as well as in patients with AATD receiving and not receiving augmentation therapy.

This study demonstrates statistically significant decreases in levels of DI in plasma in AATD patients on long term intravenously administered AAT replacement. The measurements of DI in BALF from 8 of 10 patients demonstrated statistically significant reductions in levels. This result suggests that intravenous (I.V.) augmentation therapy is reducing elastin degradation specifically in the lung, a desired result of therapy.

The reductions in plasma levels of DI in populations of patients with AATD receiving and not receiving augmentation therapy, as well as specific individuals before and after receiving AAT therapy, is consistent with a decrease of elastin degradation systemically and a systemic anti-inflammatory effect.

The significant reductions of DI in BALF in patients receiving aerosol administration of AAT is an indication that nebulized AAT is reducing the activity of neutrophil elastase in the lung per se and suggests that this route of administration may be effective therapy in AATD. The reductions in plasma levels of DI in this limited number of patients were not as consistent with aerosol administration as the reductions of DI in plasma of patients receiving I.V. administration. The more inconsistent reduction in DI levels in plasma with aerosol administration may be related to the lower weekly total dose of AAT being administered by aerosol, compared with the intravenous dose. A 70 kilogram body weight subject received a 43% lower weekly dose by aerosol than I.V. The positive correlation of DI levels in plasma and BALF sampled at the same time of 12 weeks of therapy (FIG. 12) is consistent with the levels of DI in lung positively contributing to levels in plasma.

The reduction in the percent of free DI in urine suggests less elastase degradation of elastin fragments in vivo prior to excretion, which would also be consistent with an anti-inflammatory effect of AAT augmentation. (Rodriguez et al., 1979). This result, obtained in only 5 patients with available specimens, was slightly below statistical significance (p=0.0625).

It is noteworthy that the 50 patients receiving augmentation therapy had plasma levels still above the normal range of DI. This result raises the prospect that higher doses of augmentation therapy may achieve even more effective reductions in elastase activity in AATD individuals.

Analysis of levels of DI in 43 control subjects without lung disease or alpha-1 antitrypsin deficiency (AATD) allowed us to relate plasma levels of DI to age. There was a positive correlation of increase in plasma DI levels with advancing age in non-AATD normals, as shown in FIG. 14 (r²=0.1705 and p=0.0059). These data included smokers as well as non-smoking normal subjects. As shown, there are positive correlations of plasma DI level with age in the AATD cohorts receiving and not receiving augmentation.

The information from patients in these cohorts obtained from a questionnaire on their history of smoking was considered not reliable enough to enter into this analysis. The patient questionnaire allowed individuals with a history of having smoked 20-pack-years of cigarettes in their lifetimes or less to be categorized as non-smokers. When patients started and stopped smoking could not be determined and there was no information to rule out second-hand smoke exposure. Accordingly, we analyzed each cohort with respect to age, without separating tobacco smoke-exposed and those not exposed.

The increase in plasma levels of DI related to age in all three cohorts of subjects in this study is evidence that age is associated with increased degradation of body elastin. The exact mechanisms causing this effect are unclear. However, these data in human subjects are consistent with such evidence in senescence-accelerated mice (Atanasova et al., 2010) and in studies of human aortic elastin showing reduced aorta elasticity with aging and a progressive reduction of the cross-links of aortic elastin in the aging subject. (Watanabe et al., 1996). Increased oxidation of elastin with age may play a role since it has been shown in vitro that oxidized elastin is subject to increased degradation by elastases. (Cantor et al., 2006; Umeda et al., 2011) Positive correlations of plasma levels of DI with age have been shown in 2 recent studies also using Mass spectrometric analytical methods. (Lindberg et al., 2012; Huang et al., 2012).

The mean value of plasma DI in the normal subjects in this study (0.22 ng/ml±0.04 SD) is slightly higher than the mean normal value of plasma DI in a previous publication (Ma et al., 2007) in 13 subjects (8 male, 5 female) (0.19 ng/ml±0.01 SD). This difference may be related to the strict exclusion of smokers or second-hand smoke exposed subjects in the prior study.

A previous study (Stockley et al., 2002) demonstrated an anti-inflammatory effect of AAT augmentation therapy based on reduced levels of leukotriene B-5, interleukin-8 and neutrophil elastase in sputum, after weeks of supplementation therapy with I.V. AAT. This result suggests that in addition to a direct inhibition of neutrophil elastase, augmentation therapy may reduce elastin degradation by reducing elastase production by neutrophils and macrophages through an additional anti-inflammatory effect.

The positive results with measurements in plasma and BALF indicate that these body fluids can reflect changes in systemic elastin degradation as well as organ systems. The use of HPLC MS/MS technology has made measurements in these body fluids other than urine more sensitive and precise, and deserves further application as biomarkers to evaluate potential therapies in COPD.

The plasma, BALF and urine for analysis in this study were stored in the deep frozen state (−20° c.) for several years and there can be concern that such prolonged storage, even in the frozen state, could affect the content of DI. We believe there is ample evidence that this is not the case: 1) The levels of DI in the normal subjects and in the AATD cohorts were in the same range as had been obtained from prior studies on freshly sampled plasma from similar cohorts (Ma et al., 2007; Ma et al., 2003; Ma et al., 2011); 2) we have performed repeated analyses of frozen samples of plasma and urine stored in our laboratory for over 6 years and the quantitation of DI by HPLC/MS/MS is unchanged; 3) the purpose of this study was to determine differences in the DI levels before and after administration of augmentation therapy, so before and after samples are exposed to the same storage conditions; and 4) the chemical bonding of DI is stable and, thus far, no chemical mechanisms have demonstrated molecular degradation of DI in body fluids. Acid hydrolysis of samples has been shown not to degrade DI. (Ma et al., 2011).

This study raises the consideration that AATD patients not receiving augmentation therapy may have higher DI levels in plasma because of a differing severity of disease. However, the mean FEV₁ level in patients receiving augmentation therapy was 41.3% (S.D. 22.1) while the mean FEV₁ in those not those not receiving augmentation was 72.7% (S.D. 29.3). Prior studies demonstrate that DI levels in urine and plasma are higher with increasing severity of disease as indicated by the FEV₁, which is counter to this premise. (Lindberg et al., 2012; Hung et al., 2012; Fregonese et al., 2011).

The positive correlation of age and plasma levels of DI in these patient cohorts does not have an effect on the concluding results obtained in this study. In that regard, the patients receiving augmentation therapy had a higher mean age than the patients not on augmentation and yet their mean levels of DI were statistically significantly lower.

### Example 8

This study was structured to evaluate 49 patients with severe AATD and normal lung function. Individuals in this study were not on augmentation therapy. The baseline quantitative computed tomography (QCT) defines the percentage of lung less than −910 Hounsfield units and the median value will be chosen to divide subjects into those with greater lung density and those with less lung density. Patients had study visits every six months for two years with a final visit at the end of year three. QCTs were monitored at high and low resolution at every visit and expiratory QCTs at visit 1 and at the end of year 2. This study can pilot the development of more accurate assessment of lung tissue loss and may improve the understanding of the lung destruction in AATD.

### Results

Each subject had an analysis of 6 samples of plasma or serum over a period of 3 years. There was a remarkable stability of the biomarker over this 3-year period. The level of DI at baseline was 0.31±0.07 ng/ml for all 49 subjects and 0.30±0.07 ng/ml at the final reading. These levels are significantly elevated over the normal of 0.19±0.03 ng/ml. In 30 subjects, the DI levels were reduced below the 3-year level baseline with a reduction of 11.11% (range of 2.20-36.70%), and in 19 subjects, the DI levels were increased over the 3-year period with an average increase of 15.87% (range of 0.61-49%). For 31 female subjects, the mean level of DI at baseline was 0.32 ng/ml (range of 0.21-0.47) and at 3 years was the mean 0.32 ng/ml (range of 0.19-0.46). For 18 males, the baseline DI level was 0.29 ng/ml (range of 0.20-0.36) and 0.29 ng/ml (range of 0.19-0.36) at 3 years.

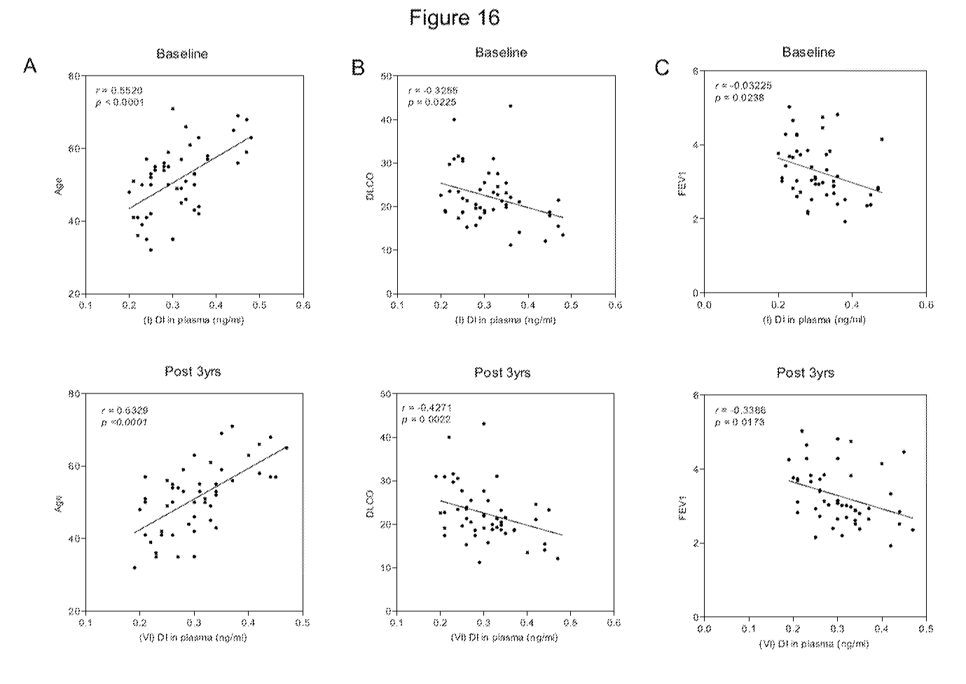

Statistically significant correlations with levels of DI were present for forced expiratory volume in one second (FEV₁), diffusing capacity of the lung for carbon monoxide (DL_{CO+}), and age, as seen in FIG. 16. Not significant correlations were found for baseline QCT density values, the ratio of FEV₁ to forced vital capacity (FEV₁/FVC) and total lung capacity.

These results suggest that the stability of these levels of DI in plasma over a 3-year interval could provide a useful baseline for the evaluation of the effectiveness of therapeutic agents, as has already been shown for AAT augmentation therapy. (Ma et al., 2013).

As has been shown above, there is a positive correlation of levels of DI in plasma with age. This correlation is also shown in this study.

It is noteworthy that plasma levels of DI were elevated above our laboratory normal for all patients at baseline and after 3 years of follow up. The significance of these elevations is that elastin degradation in the whole body is increased above normal. It is possible, and even likely, recognizing that pulmonary emphysema is a consequence of the reduced inhibitory capacity for neutrophil elastase in this population, that this degradation of elastin is occurring in the lung. Also, from previously published studies (Ma et al., 2006; Ma et al., 2013), it is recognized that patients with AATD with increased DI levels in plasma, have significant amounts of DI in BALF and in sputum. The elevation of DI in plasma in this early disease cohort signifies that the pathogenic process associated with AATD emphysema had already begun and could be a target for therapy.

Unexpected is the closeness of levels of DI for a given individual over the 3-year period of observation, which suggests that the factors determining the plasma level of DI remain consistent over time. These results also suggest that the stability of these DI levels over a 3-year interval could provide a useful baseline for the evaluation of the potential effectiveness of therapeutic agents, as has already been shown for AAT augmentation therapy.

There is a positive correlation of levels of DI in plasma with age. (Ma et al., 2013). This correlation is shown in this study with a percent increase of 20% over a 50-year interval over age 25. Previous studies have indicated that oxidation of elastin in vitro increases its susceptibility to degradation. Conceivably, increased oxygenation of elastin in vivo may occur with age and may be one possible cause for the effects of age on body elastin degradation.

Plasma samples are undergoing further analysis for levels of free (unconjugated) DI as an enhanced index of active elastin degradation.

### DOCUMENTS

AKAGAWA, M., SUYAMA, K. Mechanism of formation of elastin crosslinks. Connect. Tissue Res., 2000. 41: p. 131-141.
ALBARBARAWI, O., BARTON, A., LIN, Z., TAKAHASHI, E., BUDDHARAJU, A., BRADY, J., MILLER, D., PALMER, C. N. A., HUANG, J. T. Measurement of urinary total desmosine and isodesmosine using isotope-dilution liquid 18 chromatography-tandem mass spectrometry. Anal. Chem., 2010. 82: 745-3750.
ANNOVAZZI, L., VIGLIO, S., GHEDUZZI, D., PASQUAIL-RONCHETTI, I., ZANONE, C., GETTA, G., LADOROLA, P. High levels of desmosines in urine and plasma of patients with pseudoxanthoma elasticum. Eur. J. Clin. Invest., 2004. 34: 156-164.
ANNOVAZZI, L., VIGLIO, S., PERANI, E., LUISETTI, M., BARANIUK, J., CASADO, B. et al. Capillary electrophoresis with laser-induced fluorescence detection as a novel sensitive approach for the analysis of desmosines in real samples. Electrophoresis, 2004. 25: 683-691.
ATANASOVA M, Konova E, Georgieva M et al. Age-related changes of anti-elastin antibodies in senescence-accelerated mice. Gerontology 2010; 56:310-318.
BODE, D. C., PAGANI, E. D., CUMINSKEY, R., VON ROEMELING, R., HAMEL, L., SILVER, P. J. Comparison of urinary desmosine excretion in patients with chronic obstructive disease or cystic fibrosis. Pul. Pharmacol. Ther., 2000. 13: 175-180.
BOSCHETTO, P., QUINTAVALLE, S., ZENI, E., LEPROTTI, S., POTENA, A., BALLERIN, L. et al. Association between markers of emphysema and more severe chronic obstructive pulmonary disease. Thorax, 2006. 61(17): 1037-1042.
BOUTIN, M., AHMAD, I., JAUHIAINEN, M., LACHAPELLE, N., RONDEAU, C., ROY, J., THIBAULT, P. NanoLC-MS/MS analysis of urinary desmosine, hydroxylysylpyridinoline and lysylpyridinoline as biomarkers for chronic graft-versus-host disease. Anal. Chem., 2009. 81: 9454-9461.
BOUTIN, M., BERTHELETTE, C., GERVAIS, F. G., SCHOLAND, M. B., HOIDAL, J., LEPPERT, M. F., BATEMN, K. P., THIBAULT, P. High-sensitivity nanoLC-MS/MS analysis of urinary desmosine and isodesmosine. Anal. Chem., 2009. 81: 1881-1887.
BRANTLY M L, Paul L D, Miller B H, Falk R T, Wu M, Crystal R G: Clinical features and history of the destructive lung disease associated with alpha-1 antitrypsin deficiency of adults with pulmonary symptoms. American Review of Respiratory Disease 1988, 138:327-36.
CANTOR J O, Shteyngart B, Cerreta J M et al. Synergistic effect of hydrogen peroxide and elastase on elastin injury in vitro. Experimental Biology and Medicine 2006; 231(1):107-111.
COCCI, F., MINIATI, M., MONTI, S., CAVARRA, E., GAMBELLI, F., BATTOLLA, L. et al. Urinary desmosine excretion is inversely correlated with the extent of emphysema in patients with chronic obstructive pulmonary disease. Int. J. Biochem. Cell Biol., 2002. 34: 594-604.
CUMISKEY, W. R., PAGANI, E. D., BODE, D. C. Enrichment and analysis of desmosine and isodesmosine in biological fluids. C. J. Chromatogr B, 1995. 668: 199-207.
DIRKSEN A, Dijkman J H, Madsen F, Stoel B, Hutchison D C, Ulrik C S, Skovgaard L G, Koj-Gensen A, Rudolphus A, Seersholm N, Vrooman H A, Reiber J H, Hansen N C, Heckscher T and Viskum K, Stolk J: A randomized clinical trial of alpha (1)-antitrypsin augmentation therapy. AJRCCM 1999, 160(5 Pt 1):1468-72.
DIRKSEN A, Friis M, Olesen K P, Skovgaard L T and Sorensen: Progress of emphysema in severe alphs-1 antitripsin deficiency as assessed by annual C T. Acta Radiologica 1997, 38:826-32.
DOLLERY, C. M., OWEN, C. A., SUKOVA, G. A., KRETTEK, A., SHAPIRO, S. D., LIBY, P. Neutrophil elastase in human atherosclerotic plaques: production by macrophages. Circulation, 2003. 107: p. 2829-2836.
FIORENZA, D., VIGLIO, S., LUPI, A., BACCHESCHI, J., TINELLI, C., TRISOLINI, R. et al. Urinary desmosine excretion in acute exacerbations of COPD: a preliminary report. Respir. Med., 2002. 96: 110-114.
FREGONESE L, Ferrari F, Fumagalli F et al: Long-term variability of desmosine/isodesmosine as biomarker in alplha-1 antitrypsin deficience related COPD. COPD 2011, 8:329-333.
GALIS, Z. S., SUKHOVA, G. K., LARK, M. W., LIBY, P. Increased expression of matrix metalloproteinases and matrix degrading activity in vulnerable regions of human atherosclerosis plaque. J. Clin. Invest., 1994. 94: p. 2493-2503.
GOTTLIEB D J, Luisetti M, Stone P J et al: Short-term supplementation therapy does not affect elastin degradation in severe alpha-antitrypsin deficiency. The American-Italian AATD Study Group. AJRCCM 2000, 162:2069-72.
HUANG J T-J, Chaudhuri R, Albarbarawi O, Barton A, Grievson C, Rauchhaus P, Weir C W, Messow M, Stevens N, McSharry C, Feuerstein G, Mukhopadhyay S, Brady J, Palmer C A N, Miller D and Thomson N C: Clinical validity of plasma and urinary desmosine as biomarkers for chronic obstructive pulmonary disease. Thorax 2012, 67:502-08.
KING G S, Mohan V S, Starcher B C: Radioimmunoassay for desmosine. Connect Tissue Res 1980, 7:263-67.
LINDBERG, C. A., ENGSTROM, G., GERHARDSSON D E VERDIER, M., NIHLEN, U., ANDERSON, M., FORSMAN-SEMB, K., Svartengren, M. Total desmosine in plasma and urine correlate with lung function. Eur. Respir. J., 2012. 39: 839-845.
LUISETTI, M., M A, S., IADAROLA, P., STONE, P. J., VIGLIO, S., CASADO, B., LIN, Y. Y., SNIDER, G. L., TURINO, G. M. Desmosine as a biomarker of elastin degradation in COPD: Current status and future directions. Eur. Respir. J., 2008. 32: 1146-1157.
LUISETTI, M., STURANI, C., SELLA, D., MADONINI, E., GALAVOTTI, V., BRUNO, G. et al. MR889, a neutrophil elastase inhibitor, in patients with chronic obstructive pulmonary disease: a double-blind, randomized, placebo-controlled clinical trial. Eur. Respir. J., 1996. 9: 1482-1486.
M A S, Lin Y Y, He J, Rouhani F N, Brantly M and Turino G M: Alpha-1 Antitrypsin Augmentation Therapy and Biomarkers of Elastin Degradation. J COPD 2013, (10(4):473-81.
M A, S., LIEBERMAN, S., TURINO, G. M., LIN, Y. Y. The detection and quantitation of free desmosine and isodesmosine in human urine and their peptide-bound forms in sputum. Proc. Natl. Acad. Sci. U.S.A., 2003. 100: 12941-12943.
M A, S., LIN, Y. Y., TURINO, G. M. Measurements of desmosine and isodesmosine by mass spectrometry in COPD. Chest, 2007. 131: 1363-1371.
M A, S., TURINO, G. M., LIN, Y. Y. Quantitation of desmosine and isodesmosine in urine, plasma, and sputum by LC-MS/MS as biomarkers for elastin degradation. J. Chromatogr. B, 2011. 879: 1893-1898.
MANNINO, D. M., BUIST, A. S. Global burden of COPD: risk factors, prevalence, and future trends. Lancet, 2007. 370: 765-773.
MARQUE, V., KIEFFER, P., GAYRAUD, B., LARTAUD-IDJOUADIENE, I., RAMIREZ, F., ATKINSON, J. Aortic wall mechanics and composition in a transgenic mouse model of Marfan syndrome. Arterioscler. Thromb. Vasc. Biol., 2001. 21: 1184-1189.
MCCLINTOCK, D. E., STARCHER, B., EISNER, M. D., THOMPSON, B. T., HAYDEN, D. L., CHURCH, G. D. et al. Higher urine desmosine levels are associated with mortality in patients with acute lung injury. Am. J. Physiol. Lung Cell Mol. Physiol., 2006. 291: L566-571.
MECHAM, R. P. Elastin fibers in the lung. Scientific Foundation, Philadelphia, Lippincott-Raven, 1997, p. 729-736.
MININO, A. M., X U, J., KOCHANEK, K. D. National Vital Statistics Reports, 2010. 59: 1-52.
RABE, K. F., HURD, S., ANZUETO, A. et al. Global strategy for the diagnosis, management, and prevention of chronic obstructive pulmonary disease: GOLD executive summary. Amer. J. Resp. Crit. Care Med., 2007. 176: 532-555.
RENNARD S I, Basset G, Lecossier D, O'Donnell K M, Pinkston P, Martin P G and Crystal R: Estimation of volume of epithelial lining fluid recovered by lavage using urea as marker of dilution. J Appl Physiol 1986, 60(2):532-38.
RENNARD, S., TURINO, G. M., LIN, Y. Y., H E, J., CANTOR, J. O., M A, S. Elastin degradation: An effective biomarker in COPD. COPD, 2012. 9: 1-4.
RODRIGUEZ J R, Seals J E, Radin A, Lin J S, Mandl I, Turino G M: Neutrophil lysosomal elastase activity in normal subjects and in patients with chronic obstructive pulmonary disease. Am Rev Respir Dis 1979, 119:409-17.
ROSENBLOOM, J. Elastin: biosynthesis, structure, degradation, and role in disease processes. Connect. Tissue Res., 1982. 10: p. 73-91.
ROTH, M. D., CONNETT, J. E., D'ARMIENTO, J. M., FORONJY, R. F., FRIEDMAN, P. J., GOLDIN, J. G., LOUIS, T. A., MAO, J. T., MUINDI, J. R., O'CONNOR, G. T., RAMSDELL, J. W., RIES, A. L. I., SCHARF, S. M., SCHLUGER, N. W., SCIURBA, F. C., SKEANS, M. A., WALTER, R. E., WENDT, C. H., WISE, R. A. Feasibility of retinoids for the treatment of emphysema study. Chest, 2006. 130: 1334-1345.
SANDBERG, L. B., SOSKEL, N. T., LESLIE, J. G. Elastin structure, biosynthesis, and relation to disease states. NEJM, 1981. 304: p. 566-579.
SCHRIVER, E. E., DAVISON, J. M., SUTCLIFFE, M. C., SWINDELL, B. B., GORDON, B. Comparison of elastin peptide concentration in body fluids from healthy volunteers. Am. Rev. Respir. Dis., 1992. 145: 762-766.
SCHWARTZ, E., CRUICKSHANK, F. A., LEBWOHOL, M. Determination of desmosine in elastin-related skin disorders by isocratic HPLC. Exp. Mol. Pathol., 1990. 52: 63-68.
SHIMADA, W., BOWMAN, N. R., ANWAR, R. A. An approach to the study of the structure of desmosine and isodesmosine containing peptides isolated from the elastase digest of elastin. Biochem. Biophys. Res. Commun., 1969. 37: p. 191-197.
SPENCER L T, HUMPHRIES J E and BRANTLY M L (for the Transgenic Human Allpha1-Antitrypsin Study Group): Antibody response to aerosolized transgenic human alpha₁ antitrypsin. NEJM 2005, 352:19.
STARCHER, B., GREEN, M., SCOTT, M. Measurement of urinary desmosine as an indicator of acute pulmonary disease. Respiration, 1995. 62: 252-257.
STOCKLEY R A, Bayley D L, Unsal I and Dowson L J: The Effect of Augmentation Therapy on Bronchial Inflammation in Alpha-1 Antitrypsin Deficiency. AJRCCM 2002, 165:1494-98.
STOLK, J., VELDHUISEN, B., ANNOVAZZI, L., ZANONE, C., VERSTEEG, E., VAN KUPPEVELT, T. et al. Short-term variability of biomarkers of proteinase activity in patients with emphysema associated with type Z1-antitrypsin deficiency. Respir. Res., 2005. 6: 47-53.
STOLLER J K, Rouhani F, Brantly M et al: Biochemical efficacy and safety of a new pooled human plasma alpha-1 antitrypsin, Respitin. CHEST 2002, 122:66-74.
STONE P J, Bryan-Rhafdi J, Lucey E C, et al: Measurement of urinary desmosine by isotope dilution and high performance liquid chromatography. Correlation between elastase-induced air-space enlargement in the hamster and elevation of urinary desmosine. Am Rev Respir Dis 1991, 144:2844-90.
STONE P J, Morris L A 3^{rd}, Franzblau C, Snider G L: Preliminary evidence that augmentation therapy diminishes degradation of cross-linked elastin in alpha-1-antitrypsin-deficient humans. Respiration 1995, 62:76-79.
STONE, P. J., GOTTLIEB, D. J., O'CONNOR, G. T., CICCOLELLA, D. E., BREUER, R., BRYAN-RHADFI, J., SHAW, H. A., FRANZBLAU, C., SNIDER, G. Elastin and collagen degradation products in urine of smokers with and without chronic obstructive pulmonary diseases. Am. J. Respir. Crit. Care Med., 1995. 151: 952-959.
STONE, P. J., KONSTAN, M. W., BERGER, M, DORKIN, H. L., FRANZBLAU, C., SINDER, G. L. Elastin and collagen degradation products in urine of patients with cystic fibrosis. Am. J. Respir. Crit. Care Med., 1995. 152: 157-162.
TENHOLDER, M. F., RAJAGOPAL, K. R., PHILLIPS, Y. Y., DILARD, T. A., MUNDIE, T. G., TELLIS, C. J. Urinary desmosine excretion as a marker of lung injury in the adult respiratory distress syndrome. Chest, 1991. 100: 1385-1390.
THOMAS, J., ELDSON, D. F., PATRIDGE, S. M. Degradation products from elastin: partial structure of 2 major degradation products from cross-linkages in elastin. Nature, 1963. 200: p. 651-652.
TURINO, G M: Editorial: COPD and biomarkers: The search goes on. Thorax 2008, 63:1032-34.
UMEDA, H., AIKAWA, M., LIBBY, P. Liberation of desmosine and isodesmosine as amino acids from insoluble elastin by elastolytic proteases. Biochem. Biophys. Res. Commun., 2011. 411: p. 281-286.
USUKI, T., YAMADA, H., HAYASHI, T., YANUMA, H., KOSEKI, Y., SUZUKI, N., MASUYAMA, Y., LIN, Y. Y. Total synthesis of COPD biomarker desmosine that crosslinks elastin. Chem. Commun., 2012. 48: 3233-3235.
VIGLIO, S., LADAROLA, P. P., LUPI, A., TRISOLINI, R., TINNELI, C., BALBI, B., GRASSI, V., WORLITZSCH, D., DORING, G., MELONI, F. et al. MEKC of desmosine and isodesmosine in urine of chronic destructive lung disease patients, Eur. Respir. J., 2000. 15: 1039-1045.
VIGLIO, S., ZANABONI, G., LUISETTI, M., TRISOLINI, R., GRIMM, R., CETTA, G., IADAROLA, P. J. Micellar electrokinetic chromatography for the determination of urinary desmosine and isodesmosine in patients affected by chronic obstructive pulmonary disease. J. Chromatogr B, 1998. 714: 87-98.
WATANABE M, Sawai T, Nagura H et al. Age-related alteration of cross-linking amino acids of elastin in human aorta. Tohoku J. Exp Med 1996; 180:115-130.
WATANABE, M., SAWAI, T. Age-related alteration of cross-linking amino acids of elastin in human aorta. Tohoku J. Exp. Med., 1999. 187: p. 291-303.
WEWERS M D, Casalaro M A, Sellers S E et al: Replacement therapy for alpha-1 antitrypsin deficiency associated with emphysema. NEJM 1987, 316(17):1055-62.
YANUMA, H., USUKI, T. Total synthesis of the COPD biomarker desmosine via Sonogashira and Negishi cross-coupling reactions. Tetrahedron Lett., 2012. 53: 5920-5922.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method of measuring the amount of a marker of elastic fiber injury selected from the group consisting of desmosine, isodesmosine, and a combination thereof in a sample, comprising contacting the sample with a compound of formula (1): 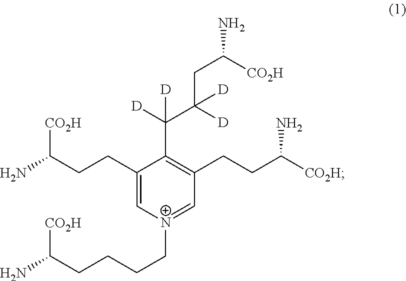 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]
and carrying out mass spectrometry on the sample containing the compound of formula (1).

2. The method according to claim 1, wherein the amount of the compound of formula (1) is pre-determined.

3. The method according to claim 1, further comprising subjecting the sample containing the compound of formula (1) to acid hydrolysis prior to mass spectrometry.

4. The method according to claim 1, wherein the sample is selected from the group consisting of connective tissue matrices, urine, plasma, sputum, bronchoalveolar lavage fluid (BALF), and combinations thereof.

5. The method according to claim 1, wherein the sample is obtained from a subject suspected of having a disease characterized by elastic fiber injury.

6. The method according to claim 5, wherein the disease is selected from the group consisting of atherosclerosis, aortic aneurysm, skin lesion, cystic fibrosis, and chronic obstructive pulmonary disease (COPD).

7. The method according to claim 6, wherein the disease is COPD.

8. The method according to claim 7, wherein the COPD is pulmonary emphysema.

9. The method according to claim 5, wherein the subject is human.

10. The method according to claim 1, wherein the amount of desmosine in the sample is calibrated in relation to the amount of the compound of formula (1).

11. The method according to claim 1, wherein the amount of desmosine and isodesmosine in the sample is calibrated in relation to the amount of the compound of formula (1).

12. The method according to claim 1, wherein the mass spectrometry is liquid chromatography mass spectrometry (LC-MS) or liquid chromatography tandem mass spectrometry (LC-MS/MS).

13. The method according to claim 1, wherein the mass spectrometry is liquid chromatography tandem mass spectrometry (LC-MS/MS).

14. A method of diagnosing whether a subject has a disease characterized by an elastic fiber injury comprising:
(a) contacting a compound of formula (1): 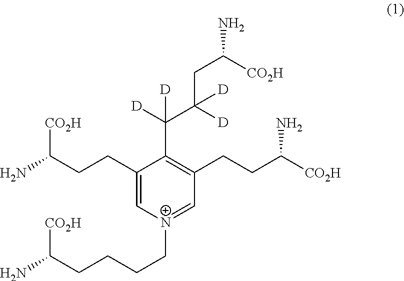 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]
with a sample obtained from the subject; and
(b) measuring, by mass spectrometry, the amount of a marker of elastic fiber injury selected from the group consisting of desmosine, isodesmosine, and a combination thereof in the sample.

15. The method according to claim 14, wherein the disease is selected from the group consisting of atherosclerosis, aortic aneurysm, skin lesion, cystic fibrosis, and chronic obstructive pulmonary disease (COPD).

16. The method according to claim 15, wherein the disease is COPD.

17. The method according to claim 14, wherein the subject is human.

18. The method according to claim 14, wherein the amount of desmosine in the sample is calibrated in relation to the amount of the compound of formula (1).

19. The method according to claim 14, wherein the amount of desmosine and isodesmosine in the sample is calibrated in relation to the amount of the compound of formula (1).

20. A method of improving the accuracy and precision of mass spectroscopy analysis of a marker of elastic fiber injury in a sample, the marker being selected from the group consisting of desmosine, isodesmosine, and a combination thereof comprising
(a) contacting a compound of formula (1): 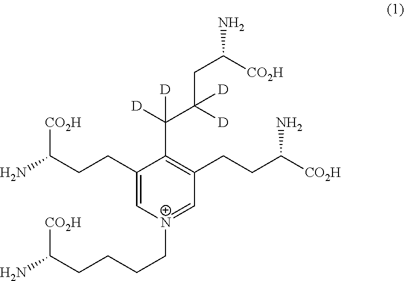 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]
with a sample from a subject suspected of having a disease characterized by elastic fiber injury;
(b) carrying out acid hydrolysis of the sample from step (a) containing the compound of formula (1); and
(c) carrying out mass spectrometry on the acid hydrolyzed sample from step (b).

21. A kit for determining, by mass spectrometry, the amount of a marker of elastic fiber injury in a sample from a subject, the kit comprising a compound of formula (1): 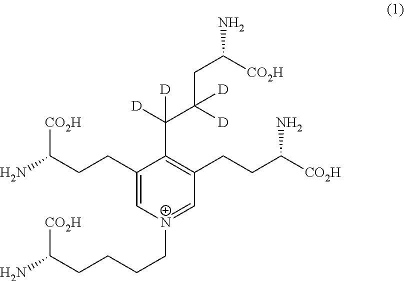 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]
and instructions for use thereof, wherein the marker of elastic fiber injury is selected from the group consisting of desmosine, isodesmosine, and a combination thereof.

22. A method for preventing the progression of the effects associated with alpha-1 antitrypsin deficiency (AATD) in a subject with normal lung function comprising
(a) measuring, by mass spectrometry, a marker of elastic fiber injury selected from the group consisting of desmosine, isodesmosine, and a combination thereof in a sample from the subject; and
(b) administering AATD augmentation therapy if the subject has a higher than a normal amount of the marker of elastic fiber injury.

23. The method according to claim 22, wherein the subject is a mammal.

24. The method according to claim 23, wherein the subject is a human.

25. The method according to claim 22, wherein the sample is contacted with a compound of formula (1): 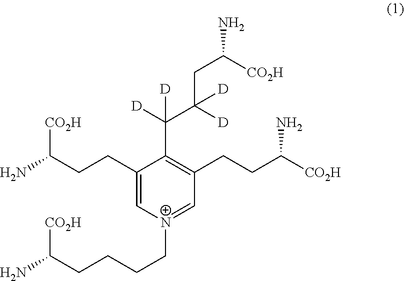 [SMILES: [2H]C([2H])(C[C@H](N)C(=O)O)C([2H])([2H])c1c(CC[C@H](N)C(=O)O)c[n+](CCCC[C@H](N)C(=O)O)cc1CC[C@H](N)C(=O)O]
prior to carrying out mass spectroscopy.

26. The method according to claim 22, wherein the sample is selected from the group consisting of connective tissue matrices, urine, plasma, serum, sputum, bronchoalveolar lavage fluid (BALF), and combinations thereof.

27. A method for detecting lung elastin degradation in a subject with normal lung function comprising measuring, by mass spectrometry, a marker of elastic fiber injury selected from the group consisting of desmosine, isodesmosine, and a combination thereof in a sample from the subject.
